(19) United States
(12) Patent Application Publication
(10) Pub. No.: US 2024/0197771 A1
(43) Pub. Date: Jun. 20, 2024

(54) COMPOSITIONS AND USES THEREOF

(71) Applicants: Harshita PANT, North Adelaide (AU); Angel LOPEZ, Medindie (AU); Mark SCHEMBRI, North Adelaide (AU)

(72) Inventors: Harshita PANT, North Adelaide (AU); Angel LOPEZ, Medindie (AU); Mark SCHEMBRI, North Adelaide (AU)

(21) Appl. No.: 18/569,597

(22) PCT Filed: Jun. 17, 2022
(86) PCT No.: PCT/AU2022/050611

§ 371 Date: Dec. 12, 2023

(30) Foreign Application Priority Data
Jun. 17, 2021 (AU) 2021901822

(51) Int. Cl. A61K 31/728; A61K 9/00; A61K 9/06; A61K 31/352; A61P 11/02; A61P 17/02

(52) U.S. Cl. CPC A61K 31/728; A61K 9/0043; A61K 9/06; A61K 31/352; A61P 11/02; A61P 17/02

(57) ABSTRACT

The present disclosure relates generally to a topical composition comprising a therapeutically effective combination of (i) cromoglycic acid, or a pharmaceutically acceptable salt thereof, and (ii) hyaluronic acid, or a pharmaceutically-acceptable salt thereof (HA), which is suitable for nasal administration for the treatment of inflammation and wounds.

### FIELD OF THE INVENTION

The present invention relates generally to compositions and uses thereof for the treatment of conditions associated with inflammation, including wound healing.

### BACKGROUND

All references, including any patents or patent application, cited in this specification are hereby incorporated by reference to enable full understanding of the invention. Nevertheless, such references are not to be read as constituting an admission that any of these documents forms part of the common general knowledge in the art, in Australia or in any other country.

Chronic rhinosinusitis (CRS) refers to inflammation of the sinonasal mucosa that usually persists for a period of greater than 12 weeks. Typical symptoms include nasal congestion or obstruction rhinorrhea, post nasal drainage, an altered sense of smell and facial discomfort. CRS is understood to affect about 5-15% of the general population, although geographical differences are evident. From a socioeconomic perspective, CRS has a significant impact on health-related quality of life and is associated with substantial healthcare and productivity costs.

The diagnosis of CRS is typically based on the presence of sinonasal symptoms that persist greater than 12 weeks, and confirmed evidence of sinus involvement, either endoscopically (e.g., by the presence of nasal polyps/mucopurulent discharge/edema in the middle meatus) or radiologically (e.g., by the presence of mucosal changes in the sinuses/ostiomeatal complex on computed tomography (CT) scans). CRS can be further subdivided into two main clinical categories based on whether nasal polyps (NP) are present or absent—chronic rhinosinusitis with nasal polyps (CRSwNP) or chronic rhinosinusitis without nasal polyps (CRSsNP). Approximately 20% of patients with CRS will present with nasal polyps. Nasal polyps in the context of CRS refers to inflammatory sinus mucosal projections that prolapse into the nasal cavities and are considered to be a manifestation of chronic inflammation causing nasal blockage, loss of sense of smell and facial discomfort. Polyp tissues are typically comprised of inflammatory cells, hyperplastic and hypersecretory mucus glands and varying degrees of oedema and fibrosis. Nasal polyps are classically associated with severe and recurrent CRS and risk of extra-sinus complications. Whilst the mechanisms underlying their development are yet to be fully elucidated, hereditary and anatomical factors, systemic and local allergy, environmental factors and infection are thought to play a role. Currently, there is no cure for CRSwNP and existing treatment involves surgical clearance of the polyps and trapped mucus by endoscopic sinus surgery, followed by anti-inflammatory treatment (typically prednisolone orally, and budesonide intranasally) and a course of antibiotics. Patient symptoms and sinus healing are monitored over time and therapy can be adjusted depending on the rate of healing. However, one of the biggest challenges in surgical treatment for nasal polyps (and sinusitis in general) is to provide an optimum microenvironment for wound healing in an already compromised sinus mucosa. The time-frame taken to achieve optimum wound healing ranges between 6-12 weeks to 6-12 months in some patients. In approximately 20% of patients with CRSwNP who have had their polyps surgically removed, complete post-operative healing is not achieved at all. Moreover, some patients continue to suffer from persistent or recurrent nasal polyps, despite currently available anti-inflammatory treatments (e.g., steroids, non-steroidals, Montelukast and long-term macrolides, including Azithromycin). A proportion of patients are also non-responsive to steroid treatment or have co-morbidities where steroid treatment is contraindicated. Hence, treatment options are limited in this cohort, some of which require repeat surgery earlier. Therefore, whilst surgical resection of nasal polyps remains the treatment of choice for patients with CRSwNP, it carries risks for intra-operative serious complications (e.g., injury to orbit or skull base) and risk of post-operative complications arising from scar formation or unsatisfactory wound healing. In some patients, surgery is contraindicated due to co-morbid conditions that pose an anaesthetic risk. The goal of surgery in the treatment of CRSwNP is to remove the inflammatory tissues, open up the sinuses widely to allow for post-operative topical medical therapy. However, surgery alone does not cure nasal polyposis and the majority of patients will require some form of ongoing post-operative medical therapy with, for example, post-operative intranasal application of saline, corticosteroids or antihistamines. In some patients who have undergone sinus surgery to remove nasal polyps, topical therapies such as these are ineffective and these patients will often be prescribed oral corticosteroid treatment. Moreover, as stated above, a proportion of patients are non-responsive to oral corticosteroid treatment and nasal polyps return within a few months of surgery, requiring repeat surgery, typically within a year for symptomatic relief. Indeed, studies have shown that 28-35% of patients with CRSwNP will develop recurrent polyposis after endoscopic ethmoidectomy or polypectomy, necessitating repeat surgery (see Dalziel et al., 2003, Health Technol Assess. 7(17):iii, 1-159). Other forms of treatment prescribed for patients with CRSwNP, include intranasal and/or systemic corticosteroids, leukotriene inhibitors, saline irrigation and antibiotics. However, such treatments are largely unsatisfactory. For example, antibiotics are not effective therapy for nasal polyps, but are often prescribed for infective exacerbations in CRSwNP. This can require repeated courses and systemic administration, which increases the risk of developing antibiotic resistance and secondary yeast infections. Whilst corticosteroids can reduce nasal polyp size, they are not curative. Chronic use of corticosteroids is also associated with severe side effects, such as hypertension, increased susceptibility to infection and bruising, weight gain, osteoporosis, joint damage and hyperglycemia. Short-term use of corticosteroids is also associated with significant risks, as discussed by Waljee A K et al. (2017) BMJ 357:j1415). Intranasal corticosteroids can also cause thinning of the nasal mucosa with subsequent bleeding. Studies have also shown non-surgical intervention to be largely ineffective in some patients with CRSwNP. For instance, as reported by Rimmer et al. (2014; “Surgical versus medical interventions for chronic rhinosinusitis with nasal polyps (Review)”; The Cochrane Collaboration), there is very little evidence showing the effectiveness of surgery versus medical treatment in adults with CRSwNP, noting that the data do not show that one treatment is better than another in terms of patient-reported symptom scores and quality of life measurements. This is particularly evident in patients with CRSwNP. The differences in nasal polyp severity, poor response to treatment and nasal polyp recurrence in CRSwNP may be attributed, at least in part, to the heterogeneity of the mechanisms underlying the pathogenesis and progression of nasal polyps. In any event, currently there is no cure for nasal polyps and the current medications used to control nasal polyps, including nasal polyp recurrence following surgical resection, carry significant side-effects. Hence, there remains an urgent need for new and improved treatment options that are effective for treating conditions associated with inflammation, including inflammation of the nasal mucosa, as well as for promoting wound healing and inhibiting the recurrence of nasal polyps following surgical resection thereof.

### SUMMARY OF THE DISCLOSURE

In an aspect disclosed herein, there is provided a topical composition comprising a therapeutically effective combination of (i) cromoglycic acid, or a pharmaceutically-acceptable salt thereof, and (ii) hyaluronic acid, or a pharmaceutically-acceptable salt thereof (HA).

In another aspect disclosed herein, there is provided a device for the topical delivery of the composition described herein. In an embodiment, the device is configured for the intranasal delivery of the composition described herein.

In another aspect disclosed herein, there is provided a method of treating a condition associated with inflammation, the method comprising administering to a subject in need thereof the composition described herein. In an embodiment, the condition is associated with inflammation of the nasal mucosa.

In another aspect disclosed herein, there is provided a method of promoting wound healing, the method comprising administering to a subject in need thereof the composition described herein. In an embodiment, the method is for promoting wound healing of the nasal mucosa.

In another aspect disclosed herein, there is provided use of a therapeutically effective combination of (i) cromoglycic acid, or a pharmaceutically-acceptable salt thereof, and (ii) hyaluronic acid, or a pharmaceutically-acceptable salt thereof (HA), in the manufacture of a medicament for treating a condition associated with inflammation in a subject in need thereof. In an embodiment, the condition is associated with inflammation of the nasal mucosa. In an embodiment, the medicament is formulated for topical administration to the subject in need thereof.

In another aspect disclosed herein, there is provided use of a therapeutically effective combination of (i) cromoglycic acid, or a pharmaceutically-acceptable salt thereof, and (ii) hyaluronic acid, or a pharmaceutically-acceptable salt thereof (HA), in the manufacture of a medicament for promoting wound healing in a subject in need thereof. In an embodiment, the condition is associated with inflammation of the nasal mucosa. In an embodiment, the medicament is formulated for promoting wound healing of the nasal mucosa in the subject in need thereof. In an embodiment, the medicament is formulated for topical administration to the subject in need thereof. In an embodiment, the medicament is formulated for intranasal administration to the subject in need thereof.

In another aspect disclosed herein, there is provided a topical composition for use in treating a condition associated with inflammation in a subject in need thereof, the composition comprising a therapeutically effective combination of (i) cromoglycic acid, or a pharmaceutically-acceptable salt thereof, and (ii) hyaluronic acid, or a pharmaceutically-acceptable salt thereof (HA). In an embodiment, the condition is associated with inflammation of the nasal mucosa. In an embodiment, the composition is formulated for intranasal administration to the subject in need thereof.

In another aspect disclosed herein, there is provided a topical composition for use in promoting wound healing in a subject in need thereof, the composition comprising a therapeutically effective combination of (i) cromoglycic acid, or a pharmaceutically-acceptable salt thereof, and (ii) hyaluronic acid, or a pharmaceutically-acceptable salt thereof (HA). In an embodiment, the composition is for use in promoting wound healing of the nasal mucosa in the subject in need thereof.

### BRIEF DESCRIPTION OF THE FIGURE

FIG. 1 shows of the degree of healing of the nasal polyp sinus in nine patients following endoscopic sinus surgery to clear the nasal polyps and open all paranasal sinuses on both sides. Patient 1 had treatment (SCG+HA (PureRegen™)) applied to both sinus cavities at end of surgical procedure. Patients 2 through 9 had treatment gel (SCG+HA) applied to one side, and vehicle (HA; PureRegen™, alone) applied to the other side as a control. Sinus cavities were inspected with a 2.9 mm rigid sinoscope at each post-operative appointment (X-axis), and treatment and control sides were scored using a modified Lund Kennedy Sinus Endoscopy Scoring System (Y-axis).

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present disclosure, the following terms are defined below.

The articles “a” and “an” are used herein to refer to one or to more than one (i.e. to at least one) of the grammatical object of the article. By way of example, “an element” means one element or more than one element. Similarly, “a peptide” means one peptide or more than one peptide.

As used herein, the term “about” refers to a quantity, level, value, dimension, size, or amount that varies by as much as 10% (e.g., by 10%, 9%, 8%, 7%, 6%, 5%, 4%, 3%, 2% or 1%) to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

### Compositions and Uses Thereof

The present disclosure is predicated, at least in part, on the inventors' surprising finding that cromoglycic acid, when formulated in combination with hyaluronic acid (HA), or a pharmaceutically-acceptable salt thereof, is capable of modulating the inflammatory milieu within the nasal mucosa and thereby treat conditions associated with inflammation of the nasal mucosa, including following endoscopic surgery. Moreover, the inventors' have surprisingly found that cromoglycic acid, when formulated in combination with hyaluronic acid (HA), is therapeutically more efficacious than HA alone.

Thus, in one aspect disclosed herein, there is provided a topical composition comprising a therapeutically effective combination of (i) cromoglycic acid, or a pharmaceutically-acceptable salt thereof, and (ii) hyaluronic acid, or a pharmaceutically-acceptable salt thereof (HA).

Cromoglycic acid is also referred to interchangeably herein as cromolyn, cromoglycate and cromoglicate. The present disclosure also extends to salts (i.e., cromoglycates) and free base forms of cromoglycic acid, along with functional analogues thereof. In an embodiment, the salt form of cromoglycic acid is sodium cromoglycate (SCG), also commonly referred herein to as cromolyn sodium. Analogues of cromoglycic acid will be familiar to persons skilled in the art, illustrative examples of which are disclosed in U.S. Pat. No. 3,419,578, the contents of which are incorporated herein by reference. Unless otherwise indicated, the terms cromolyn and cromoglycic acid are used interchangeably and include pharmaceutically-acceptable salts thereof (i.e., cromoglycates).

Methods of preparing cromoglycic acid, or a pharmaceutically-acceptable salt thereof, will be known to persons skilled in the art, an illustrative example of which is described in U.S. Pat. No. 3,419,578, the contents of which are incorporated herein by reference.

As used herein, “therapeutically effective combination” refers to the combination of cromoglycic acid (or pharmaceutically-acceptable salt thereof) and hyaluronic acid, or a pharmaceutically-acceptable salt thereof (HA), whereby they provide, in combination, the desired therapeutic effect or outcome (e.g., a reduction in the recurrence of nasal polyps and/or promotion of wound healing following resection), as described elsewhere herein. Without being bound by theory, or by a particular mode of application, the hyaluronic acid, or a pharmaceutically-acceptable salt thereof (HA) is understood to potentiate the therapeutic effect of the cromoglycic acid, or pharmaceutically-acceptable salt thereof, whether on the nasal mucosa or elsewhere in the subject, insofar as the administration of the cromoglycic acid, or pharmaceutically-acceptable salt thereof, to the subject, when formulated in the absence of hyaluronic acid, or a pharmaceutically-acceptable salt thereof (HA), will have no therapeutic effect, or less of a therapeutic effect, when compared to the administration of the cromoglycic acid, or pharmaceutically-acceptable salt thereof, to the subject, when formulated in the presence of hyaluronic acid, or a pharmaceutically-acceptable salt thereof (HA). In an embodiment, the hyaluronic acid, or a pharmaceutically-acceptable salt thereof (HA), potentiates the effect of the cromoglycic acid, or a pharmaceutically-acceptable salt thereof, as determined, for example, by the rate of wound healing and/or reduction in the recurrence of nasal polyps in the subject following resection thereof.

Without being bound by theory or by a particular mode of application, it is anticipated that the desired therapeutic effect can be further controlled by adjusting the amount of cromoglycic acid, or pharmaceutically-acceptable salt thereof, in a composition with the hyaluronic acid, or a pharmaceutically-acceptable salt thereof. In this context, the amount of cromoglycic acid, or pharmaceutically-acceptable salt thereof, is referred to herein as a therapeutically effective amount, and can suitably be determined by persons skilled in the art depending on non-limiting factors such as the severity of the condition to be treated (e.g. the degree of inflammation, the size and number of wounds following nasal polyp resection), as well as the age and general health of the subject. The therapeutically effective amount of cromoglycic acid, or pharmaceutically-acceptable salt thereof, will typically fall within a relatively broad range that can be determined through routine trials by persons skilled in the art. Such therapeutically effective amounts may also be suitably adjusted to provide an optimum therapeutic response, depending, for example, on the condition to be treated.

In an embodiment, the composition described herein comprises from about 0.5% to about 20% cromoglycic acid, or pharmaceutically-acceptable salt thereof, by weight of the total weight of the composition. By “from about 0.5% to about 20%” means about 0.5%, preferably about 0.75%, preferably about 1.0%, preferably about 1.25%, preferably about 1.5%, preferably about 1.75%, preferably about 2.0%, preferably about 2.25%, preferably about 2.5%, preferably about 2.75%, preferably about 3.0%, preferably about 3.25%, preferably about 3.5%, preferably about 3.75%, preferably about 4.0%, preferably about 4.25%, preferably about 4.5%, preferably about 4.75%, preferably about 5.0%, preferably about 5.25%, preferably about 5.5%, preferably about 5.75%, preferably about 6.0%, preferably about 6.25%, preferably about 6.5%, preferably about 6.75%, preferably about 7.0%, preferably about 7.25%, preferably about 7.5%, preferably about 7.75%, preferably about 8.0%, preferably about 8.25%, preferably about 8.5%, preferably about 8.75%, preferably about 9.0%, preferably about 9.25%, preferably about 9.5%, preferably about 9.75%, preferably about 10.0%, preferably about 10.25%, preferably about 10.5%, preferably about 10.75%, preferably about 11.0%, preferably about 11.25%, preferably about 11.5%, preferably about 11.75%, preferably about 12.0%, preferably about 12.25%, preferably about 12.5%, preferably about 12.75%, preferably about 13.0%, preferably about 13.25%, preferably about 13.5%, preferably about 13.75%, preferably about 14.0%, preferably about 14.25%, preferably about 14.5%, preferably about 14.75%, preferably about 15.0%, preferably about 15.25%, preferably about 15.5%, preferably about 15.75%, preferably about 16.0%, preferably about 16.25%, preferably about 16.5%, preferably about 16.75%, preferably about 17.0%, preferably about 17.25%, preferably about 17.5%, preferably about 17.75%, preferably about 18.0%, preferably about 18.25%, preferably about 18.5%, preferably about 18.75%, preferably about 19.0%, preferably about 19.25%, preferably about 19.5%, preferably about 19.75%, or preferably about 20.0% by weight of the total weight of the composition,

In an embodiment, the composition comprises from about 0.5% to about 10.0% cromoglycic acid, or a pharmaceutically-acceptable salt thereof, by weight of the total weight of the composition. In an embodiment, the composition comprises from about 1.0% to about 10.0% cromoglycic acid, or a pharmaceutically-acceptable salt thereof, by weight of the total weight of the composition. In an embodiment, the composition comprises from about 1.0% to about 5.0% cromoglycic acid, or a pharmaceutically-acceptable salt thereof, by weight of the total weight of the composition. In an embodiment, the composition comprises from about 2.0% to about 10.0% cromoglycic acid, or a pharmaceutically-acceptable salt thereof, by weight of the total weight of the composition. In an embodiment, the composition comprises from about 3.0% to about 10% cromoglycic acid, or a pharmaceutically-acceptable salt thereof, by weight of the total weight of the composition. In an embodiment, the composition comprises from about 3.0% to about 5.0% cromoglycic acid, or a pharmaceutically-acceptable salt thereof, by weight of the total weight of the composition. In an embodiment, the composition comprises from about 5.0% to about 10.0% cromoglycic acid, or a pharmaceutically-acceptable salt thereof, by weight of the total weight of the composition. In an embodiment, the composition comprises from about 5.0% to about 8.0% cromoglycic acid, or a pharmaceutically-acceptable salt thereof, by weight of the total weight of the composition. In an embodiment, the composition comprises from about 3.0% to about 8.0% cromoglycic acid, or a pharmaceutically-acceptable salt thereof, by weight of the total weight of the composition. In an embodiment, the composition comprises about 3.0% to about 5.0% cromoglycic acid, or a pharmaceutically-acceptable salt thereof, by weight of the total weight of the composition. In an embodiment, the composition comprises about 4% cromoglycic acid, or a pharmaceutically-acceptable salt thereof, by weight of the total weight of the composition.

The present disclosure also contemplates adjunct therapies, where the composition described herein may suitably be formulated for administration with an additional therapeutic agent. Thus, in an embodiment, the composition further comprises an additional therapeutic agent. Suitable additional therapeutic agents will be familiar to persons skilled in the art, the choice of which will likely depend on the condition to be treated. In an embodiment, the additional therapeutic agent is selected from the group consisting of a steroid, an antibiotic and an antimicrobial peptide (AMP). Illustrative examples of AMP are described in de Briej et al (2018, Sci. Trans. Med., 10:eaan4044), WO 2010/149792, WO 2014/182172 and WO 2015/088344, the entire contents of which are incorporated herein by reference. In an embodiment, the AMP is a component of a fusion protein. Illustrative examples of fusion proteins comprising AMP are described in WO 2010/149792, the entire contents of which are incorporated herein by reference.

In an embodiment, the composition described herein is formulated for intranasal administration. Such forms will be familiar to persons skilled in the art, illustrative examples of which include aerosols, sprays, drops, lotions, gels, creams and ointments. In an embodiment, the composition is formulated for intranasal administration as an aerosol, as a spray, as a nasal drop, as a lotion, as a gel, as a cream or as an ointment. In an embodiment, the composition is formulated for administration as an aerosol. In an embodiment, the composition is formulated for administration as an ointment. In an embodiment, the composition is formulated for administration as a cream. In an embodiment, the composition is formulated for administration as a gel. In an embodiment, the composition is formulated for administration as a lotion. In an embodiment, the composition is formulated for administration as a nasal drop. In an embodiment, the composition is formulated for administration as a spray.

Hyaluronic acid (HA), also known as hyaluronan, is a naturally-occurring, water soluble polysaccharide, specifically a glycosaminoglycan. HA is a major carbohydrate polymer component of extracellular matrix and is widely distributed in human and non-human tissues. HA acid is a polymer of disaccharides, which are composed of D-glucuronic acid and N-acetyl-D-glucosamine, linked via alternating β-(1→4) and β-(1→3) glycosidic bonds. The disaccharides are repeatedly arranged to form an acidic linear polymucopolysaccharide. HA has excellent biocompatibility and does not cause allergic or adverse reactions when used on or in humans and animals. As used herein, and unless otherwise defined, the term HA refers interchangeably to any of its hyaluronate salts, and includes, but is not limited to, sodium hyaluronate (NaHA), potassium hyaluronate, magnesium hyaluronate, and calcium hyaluronate. In the present disclosure, one type of hyaluronic acid may be used alone, or two or more types may be used in combination.

HA can be purified from animal and non-animal sources. The origin of HA is not to be taken as limiting and, for example, those isolated and extracted from chicken combs, umbilical cords, etc., and those prepared by a fermentation method using microorganisms such as Streptococcus can be used. Commercial sources of HA are also available (e.g., PureRegen™ (BioRegen) and MeroGel™ (Medtronic Xomed Inc, Jacksonville, Fla)).

Chemically modified forms of HA may also be suitable, as long as the chemical modification does not eliminate the desired therapeutic effect. Suitable modified forms of HA will be familiar to persons skilled in the art, illustrative examples of which include hydroxypropyltrimonium hyaluronate, hydrolyzed alkyl hyaluronate (C12-13) glyceryl, propylene glycol hyaluronate, and acetylated sodium hyaluronate. The chemically modified hyaluronic acid may be used alone or in combination of two or more different forms of HA.

HA polymers may range in size from about 5,000 Da to about 20,000,000 Da. The molecular weight of HA is not particularly limited. High molecular weight HA (sometimes herein referred to as “HMW HA”), generally describes an HA having a molecular weight more than 1.8 million Daltons to about 4.0 million Daltons. For example, the high molecular weight HA may have a molecular weight of about 2.0 million Daltons. In another example, the high molecular weight HA may have a molecular weight of about 2.8 million Daltons.

Medium molecular weight HA (sometimes herein referred to as “MMW HA”) generally describes an HA having a molecular weight between 1 to 1.8 million Daltons. For example, the MMW HA may have a molecular weight of about 1.2 million Daltons. In another example, the MMW HA may have a molecular weight of about 1.6 million Daltons.

Low molecular weight HA (sometimes herein referred to as “LMW HA”) generally describes an HA having a molecular weight of less than about 1.0 million Daltons. Low molecular weight HA can have a molecular weight of between about 200,000 Daltons to less than about 1.0 million Daltons, for example, between about 200,000 Daltons to about 990,000 Daltons, for example, between about 300,000 Daltons to about 750,000 Daltons.

Hyaluronic acid can degrade into polymers of varying length. In an embodiment, the HA, or the pharmaceutically-acceptable salt thereof, has an average molecular weight from about 10 kDa to in excess of 1,000 kDa. The compositions described herein may suitably comprise an intercalated, linear, or multifractional preparation of HA. In an embodiment, the HA comprises polymer molecules that are not cross-linked. Alternatively, or in addition, the HA comprises polymer molecules that are cross-linked, such as by using a suitable cross-linking agent, an illustrative example of which includes epoxy-alkyl-sucrose. Combinations of cross-linked and non-crossed linked HA may be formulated as a monophasic gel comprising a single fluid physical phase of a gelatinous nature. In an embodiment, the HA is stable, in the absence of microbial activity, for at least 7 days at 37° C. Other suitable forms of HA are described in WO 2019/234660, the entire contents of which are incorporated herein by reference.

The HA will typically have an inherent viscosity. In an embodiment, the HA has a viscosity or dynamic viscosity that is generally consistent with gel or other viscous compositions for therapeutic applications. The viscosity or dynamic viscosity of such compositions will be familiar to persons skilled in the art. In an embodiment, the HA has a viscosity of from about 1,000 mPa/s to about 200,000 mPa/s, preferably from about 10,000 mPa/s to about 200,000 mPa/s, more preferably from 100,000 mPa/s to about 200,000 mPa/s. In an embodiment, the HA has a dynamic viscosity of from about 1,000 mPa/s to about 200,000 mPa/s, preferably from about 10,000 mPa/s to about 200,000 mPa/s, more preferably from 100,000 mPa/s to about 200,000 mPa/s. In an embodiment, the HA has a viscosity of greater than about 100,000 mPa/s. In an embodiment, the HA has a dynamic viscosity of greater than about 100,000 mPa/s.

In an embodiment disclosed herein, the HA comprises non-cross-linked hyaluronic acid. In another embodiment, the HA comprises cross-linked HA. In another embodiment, the HA comprises self cross-linked HA. In some embodiments, the HA comprises non-cross-linked and cross-linked HA. As used herein, the term “cross-linked” refers to the intermolecular bonds joining the individual polymer molecules, or monomer chains, into a more stable structure, like a gel. As such, a cross-linked glycosaminoglycan polymer will typically have at least one intermolecular bond joining at least one individual polymer molecule to another. The crosslinking of glycosaminoglycan polymers typically result in the formation of a hydrogel that will typically have a higher viscosity than a non-cross-linked polymer.

In an embodiment, the HA is a hydrogel. In an embodiment, the HA of the hydrogel are cross-linked using crosslinking agents. Suitable cross-linking agents will be familiar to persons skilled in the art, illustrative examples of which include ethylene glycol diglycidyl ether (EGDGE), 1,4-butanediol diglycidyl ether (BDDE), 1,6-hexanediol diglycidyl ether, propylene glycol diglycidyl ether, diglycidyl ether of propylene glycol, diglycidyl ether of propylene glycol, diglycidyl ether of propylene glycol; diglycerol polyglycidyl ether and EDC (1-ethyl-3-(3-dimethylaminopropyl) carbodiimide). In an embodiment described herein, the crosslinking agent is an ether-based crosslinking agent. In an embodiment, the crosslinking agent is selected from the group consisting of ethylene glycol diglycidyl ether (EGDGE), 1,4-butanediol diglycidyl ether (BDDE), 1,6-hexanediol diglycidyl ether, propylene glycol diglycidyl ether, diglycidyl ether of propylene glycol, diglycidyl ether of propylene glycol, diglycidyl ether of propylene glycol; diglycerol polyglycidyl ether and EDC (1-ethyl-3-(3-dimethylaminopropyl) carbodiimide).

The present disclosure also extends to devices configured to deliver the compositions described herein. Hence, in another aspect, there is provided a device configured for the delivery of the composition described herein. In another aspect, there is provided a device when used for the delivery of the composition described herein. In yet another aspect, there is provided a delivery device comprising the composition described herein. Suitable devices will be familiar to persons skilled in the art, illustrative examples of which include stents, nasal atomizers, spray pumps, and nebulizers. In an embodiment, the device is an intranasal device. In an embodiment, the device is configured for the intranasal delivery of the composition described herein. In an embodiment, the device is a stent. In an embodiment, the device is a nasal atomizer. In an embodiment, the device is a spray pump.

### Uses and Methods of Treatment

As noted elsewhere herein, the present disclosure is predicated, at least in part, on the inventors' surprising finding that cromoglycic acid, when formulated in combination with hyaluronic acid (HA), or a pharmaceutically-acceptable salt thereof, is capable of modulating the inflammatory milieu within the nasal mucosa and thereby can be used to treat conditions associated with inflammation, including of the nasal mucosa (e.g., following endoscopic surgery). Moreover, cromoglycic acid, when formulated in combination with hyaluronic acid (HA), is therapeutically more efficacious than HA alone.

Thus, in an aspect disclosed herein, there is provided a method of treating a condition associated with inflammation in a subject in need thereof, the method comprising administering to a subject in need thereof a composition comprising a therapeutically effective combination of (i) cromoglycic acid, or a pharmaceutically-acceptable salt thereof and (ii) hyaluronic acid, or a pharmaceutically-acceptable salt thereof (HA).

In yet another aspect disclosed here, there is provided a method of promoting wound healing, the method comprising administering to a subject in need thereof the composition described herein.

In another aspect disclosed herein, there is provided use of a therapeutically effective combination of (i) cromoglycic acid, or a pharmaceutically-acceptable salt thereof, and (ii) hyaluronic acid, or a pharmaceutically-acceptable salt thereof (HA), in the manufacture of a medicament for treating a condition associated with inflammation in a subject in need thereof.

In an embodiment, the condition associated with inflammation is a condition associated with inflammation of the nasal mucosa. In an embodiment, the medicament is formulated for intranasal administration to the subject in need thereof. In an embodiment, the condition is associated with inflammation of the sinonasal mucosa. In an embodiment, the condition is chronic rhinosinusitis (CRS). In an embodiment, the CRS is chronic rhinosinusitis with nasal polyps (CRSwNP) or chronic rhinosinusitis without nasal polyps (CRSsNP). In an embodiment, the CRS is chronic rhinosinusitis with nasal polyps (CRSwNP). In an embodiment, the composition is formulated for administration intraoperatively following endoscopic sinus surgery.

In another aspect disclosed herein, there is provided use of a therapeutically effective combination of (i) cromoglycic acid, or a pharmaceutically-acceptable salt thereof, and (ii) hyaluronic acid, or a pharmaceutically-acceptable salt thereof (HA) in the manufacture of a medicament for promoting wound healing in a subject in need thereof. In an embodiment, the wound is a wound of the nasal mucosa. In an embodiment, the medicament is formulated for intranasal administration to the subject in need thereof.

In another aspect disclosed herein, there is provided a composition comprising a therapeutically effective combination of (i) cromoglycic acid, or a pharmaceutically-acceptable salt thereof, and (ii) hyaluronic acid, or a pharmaceutically-acceptable salt thereof (HA), for use in treating a condition associated with inflammation in a subject in need thereof.

In another aspect disclosed herein, there is provided a composition comprising a therapeutically effective combination of (i) cromoglycic acid, or a pharmaceutically-acceptable salt thereof, and (ii) hyaluronic acid, or a pharmaceutically-acceptable salt thereof (HA), for use in promoting wound healing in a subject in need thereof.

The terms “treat”, “treatment”, “treating” and the like, as used herein, refer to any and all uses which remedy, or otherwise prevent, hinder, retard, inhibit, abrogate or reverse the onset or recurrence of a condition associated with inflammatory, including inflammatory of the nasal mucosa and the onset and/or recurrence of nasal polyps or one or more symptoms thereof. The terms “treat”, “treatment”, “treating” and the like do not imply that a subject is treated until total recovery or cure. In conditions that display or that are characterized by multiple symptoms, the treatment need not necessarily remedy, prevent, hinder, retard, inhibit, abrogate or reverse all of said symptoms, but may remedy, prevent, hinder, retard, inhibit, abrogate or reverse one or more of said symptoms.

In an embodiment, the condition is associated with inflammation of the nasal mucosa.

Conditions associated with inflammation of the nasal mucosa will be familiar to persons skilled in the art, illustrative examples of which include chronic rhinosinusitis (CRS), chronic rhinosinusitis with nasal polyps (CRSwNP), chronic rhinosinusitis without nasal polyps (CRSsNP), wound healing (e.g., following endoscopic surgery, including to remove a nasal polyp). In an embodiment, the condition is associated with inflammation of the sinonasal mucosa. In another embodiment, the condition is chronic rhinosinusitis (CRS). In yet another embodiment, the CRS is chronic rhinosinusitis with nasal polyps (CRSwNP) or chronic rhinosinusitis without nasal polyps (CRSsNP), more preferably chronic rhinosinusitis with nasal polyps (CRSwNP).

In another embodiment, the condition is wound healing. In this context, the composition described herein advantageously promotes wound healing, over and above the use of HA alone. In an embodiment, the method described herein comprises administering the composition intranasally to the subject. In an embodiment, the method described herein comprises administering the composition intraoperatively to the subject following endoscopic sinus surgery. In an embodiment, the method described herein comprises administering the composition intranasally and intraoperatively to the subject following endoscopic sinus surgery.

As noted elsewhere herein, cromoglycic acid, when formulated for administration in combination with hyaluronic acid (HA), is effective at improving a condition associated with inflammation, including of the nasal mucosa, over and above the administration of HA alone. Methods of measuring a therapeutic outcome, including an improvement in a condition associated with inflammation, including inflammation of the nasal mucosa, will be familiar to persons skilled in the art, illustrative examples of which include using a Lund Kennedy Sinus Endoscopy Score or a Modified Lund Kennedy Sinus Endoscopy Score, as herein described.

In an embodiment, the method or use described herein reduces inflammation of the nasal mucosa as determined by Lund Kennedy Sinus Endoscopy Score when compared to a composition comprising HA in the absence of the cromoglycic acid, or the pharmaceutically-acceptable salt thereof.

In an embodiment, the reduction in inflammation of the nasal mucosa, as determined by Lund Kennedy Sinus Endoscopy Score, is evident by at least about 20 days following administration of the composition.

In an embodiment, the reduction in inflammation of the nasal mucosa, as determined by Lund Kennedy Sinus Endoscopy Score, is evident by at least about 40 days following administration of the composition.

In an embodiment, the reduction in inflammation of the nasal mucosa, as determined by Lund Kennedy Sinus Endoscopy Score, persists for at least about 60 days following administration of the composition.

In an embodiment, the reduction in inflammation of the nasal mucosa, as determined by Lund Kennedy Sinus Endoscopy Score, persists for at least about 80 days following administration of the composition.

As noted elsewhere herein, the present disclosure also extends to methods and uses for promoting wound healing of the nasal mucosa. In an embodiment, the wound is a post-operative lesion of the sinonasal mucosa following endoscopic sinus surgery. In an embodiment, the composition is administered intraoperatively following endoscopic sinus surgery.

In an embodiment, the subject has undergone surgery to remove nasal polyps, including polyps that have developed as a consequence of chronic rhinosinusitis, also referred to as nasal polyposis. As noted elsewhere herein, “chronic rhinosinusitis” (or “CRS”) refers to inflammation of the sinonasal mucosa that persists for a period of greater than 12 weeks, characterized by one or more symptoms, non-limiting examples of which include nasal congestion, decreased or lost sense of smell, anterior and/or posterior nasal discharge, facial pain and/or headache. Patients with CRS will often have one or more of the aforementioned symptoms for a period of years. A formal diagnosis of CRS is typically made on the basis of sinus computed tomography (CT) scan and/or sinus endoscopy. Based on endoscopic evaluation, CRS can be clinically divided as CRSwNP or CRS without nasal polyps/nasal polyposis.

In certain embodiments, the methods described herein are prescribed for the long-term treatment of subjects with chronic rhinosinusitis with bilateral nasal polyposis, who have persistent signs and symptoms despite treatment with intranasal corticosteroids (INCS). In other embodiments, the methods described herein are prescribed for the long-term treatment of subjects with bilateral nasal polyposis with associated chronic rhinosinusitis who have persistent signs and symptoms despite treatment with INCS, including subjects who also suffer from allergies to pollen, dust mite allergens and domestic pet allergens.

Nasal polyps are characterized by an overgrowth of tissue in one or more of the nasal cavities and typically present as teardrop-shaped growths that prolapse from the sinuses into the nose and/or sinuses, obstructing the sinuses and nasal passages. The condition of having nasal polyps is also commonly referred to as nasal polyposis, characterized by the presence of multiple polyps in the upper nasal cavity, originating from the osteomeatal complex. Nasal polyposis is typically characterized by chronic nasal obstruction and congestion, reduction in or loss of sense of smell, anterior and posterior rhinorrhea, and facial pain. The presence or absence of nasal polyps can be confirmed by nasal endoscopy, and the presence and extent of sinus and polyp involvement can be confirmed by sinus computed tomography (CT) scans.

As used herein, the term “bilateral nasal polyps” means the presence of one or more nasal polyps at each side of the nasal cavity.

Nasal polyposis can develop as a result of a variety of conditions that will be familiar to persons skilled in the art, illustrative examples of which include sinusitis (e.g., allergic or non-allergic sinusitis), rhinitis (e.g., allergic and non-allergic rhinitis), rhinosinusitis (e.g., allergic or non-allergic rhinosinusitis), asthma (e.g., moderate-to-severe asthma), NSAID sensitivity (e.g., aspirin sensitivity), and infection, such as bacterial and fungal infection. A subject with nasal polyposis, such as a patient with chronic rhinosinusitis with bilateral nasal polyposis, may have concomitant Samter's triad (defined by presence of nasal polyps, asthma, and aspirin and NSAID sensitivity). A subject with nasal polyposis may also have concomitant asthma, and/or other Th2 systemic conditions. Bacterial infections include, for example, Staphylococcus aureus infections. A subject with nasal polyposis may have a chronic pathogenic bacterial colonization or infection, e.g., Staphylococcus aureus. In some embodiments, the subject has recurring nasal polyposis, such as may be associated with recurring sinusitis. In other embodiments, the subject has cystic fibrosis or NARES (Non-Allergic Rhinitis with Eosinophilia Syndrome). In other embodiments, the subject has a recurrence of nasal polyposis after receiving surgery to treat the nasal polyps. Risk factors for nasal polyposis will be familiar to persons skilled in the art, illustrative examples of which include genetic susceptibility, anatomic abnormality, epithelial barrier dysfunction, mucociliary impairment, infection, atopy and local and/or systemic immunologic imbalance, hyperactivity, autoimmunity or deficiency.

The methods described herein can suitably be used to treat nasal polyposis in patients who have previously undergone surgery, such as a sinus surgery, such as for treatment of nasal polyps. In an embodiment, the subject has relapsed (or recurrent) nasal polyposis after having received prior treatment for nasal polyps, such as a prior sinus surgery.

As used herein, the term “sinusitis” refers to any inflammatory condition characterized by inflammation of the paranasal sinuses, including inflammation of the maxillary, frontal, ethmoid and/or sphenoid paranasal sinuses. The methods described herein are therefore suitable for the treatment of nasal polyposis associated with acute sinusitis, sub-acute sinusitis, chronic sinusitis and recurrent sinusitis. Acute sinusitis is typically characterized by a sudden onset of cold-like symptoms such as runny, stuffy nose and facial pain that does not go away after 10 to 14 days. Acute sinusitis typically lasts less than four weeks. Sub-acute sinusitis lasts four to twelve weeks. Chronic rhinosinusitis typically lasts twelve weeks or longer, and recurrent sinusitis is characterized by sinusitis episodes that occur three or more times in one year.

Patients with chronic rhinosinusitis will often present with chronic hyperplastic eosinophilic sinusitis, which is characterized by marked inflammation of the sinuses, increased eosinophils and mixed mononuclear cells, and a relative paucity of neutrophils. Some of these patients will have one or more of associated nasal polyps, asthma, and aspirin or NSAID sensitivity. In an embodiment, the method described herein is used to treat nasal polyposis in a subject who has chronic hyperplastic eosinophilic sinusitis.

As used herein, the term “allergic rhinosinusitis” refers to rhinosinusitis that occurs when the subject's immune system responds to specific, non-infectious irritants, non-limiting examples of which include plant pollens, molds, dust mites, animal hair, industrial chemicals (including tobacco smoke), foods, medicines, and insect venom.

As used herein, the term “non-allergic rhinosinusitis” refers to rhinosinusitis that is not due to an allergic reaction, such as from colds, allergies, and tissue irritants (e.g., nasal sprays, cocaine, cigarette smoke and the like). Less commonly, sinuses can also become obstructed by tumors or growths.

The term “rhinitis” refers to an allergic response, such as to a common allergen (allergic rhinitis, e.g., perennial allergic rhinitis) or to an environmental irritant (non-allergic rhinitis). Symptoms of allergic rhinitis will be familiar to persons skilled in the art, illustrative examples of which include sneezing, stuffy or runny nose, sinus pressure, pain or throbbing in the cheeks or nose, and itching of the nose, throat, eyes and ears.

As used herein, the term “allergic rhinitis” refers to rhinitis that occurs when the subject's immune system responds to specific, non-infectious irritants, including abnormal immune responses through the production of IgE to extrinsic and intrinsic factors.

As used herein, the term “non-allergic rhinitis” (also known as vasomotor rhinitis) refers to rhinitis that is not due to an allergic reaction, that can be triggered by factors, non-limiting example of which include cigarette smoke and other pollutants, strong odors, strong chemical environments, alcoholic beverages, cold, blockages in the nose, a deviated septum, infections and over-use of medications such as decongestants and/or nasal sprays (also known as rhinitis medicamentosa). Symptoms of non-allergic rhinitis include constriction or inflammation in the nasal passages which leads to many of the same symptoms of allergic rhinitis.

As used herein, the term “rhinosinusitis” typically refers to a condition that has symptoms of both rhinitis and sinusitis. The term “rhinosinusitis” is preferred rather than “sinusitis” by many authors because it is believed that inflammation of the sinus lining is likely to also affect the nasal lining, which is continuous, hence rhinosinusitis. However, rhinitis is considered different, where rhinitis (inflammation of the lining of the nose, including inferior turbinates) is often seen in allergies but sinus involvement (e.g., thickening of sinus mucosal lining, confirmed by sinus CT scans) may not occur. Rhinosinusitis includes acute rhinosinusitis and chronic rhinosinusitis. Acute rhinosinusitis may be caused by an infection, such as a bacterial, viral or fungal infection, allergies or by a chemical irritation. Non-limiting examples of acute rhinosinusitis include cigarette-smoke-induced acute rhinosinusitis and chlorine fume-induced chronic rhinosinusitis. Nasal polyposis is most commonly associated with CRS, which is typically characterized by mucosal inflammation of the nasal cavity and paranasal sinuses with symptoms lasting more than 12 weeks. Chronic eosinophilic rhinosinusitis with nasal polyps is a condition that also typically lasts longer than 12 weeks. The underlying causes of acute sinusitis and acute rhinosinusitis may lead to chronic sinusitis or chronic rhinosinusitis if the resulting inflammation persists for, for example, more than 12 weeks. Chronic eosinophilic rhinosinusitis with nasal polyps is a condition that also typically lasts longer than 12 weeks. Chronic rhinosinusitis includes, for example, eosinophilic chronic hyperplastic rhinosinusitis.

Chronic rhinosinusitis subtypes based on proposed mechanisms of inflammation, may be further sub-characterized as superantigen-induced eosinophilic chronic sinusitis (e.g., sinusitis induced by exo- and endo-toxins produced by bacteria such as Staphylococcus aureus), allergic fungal sinusitis (e.g., non-invasive fungal sinusitis induced by fungi such as Aspergillus or Alternaria), eosinophilic mucus chronic rhinosinusitis (EMCRS), non-allergic fungal eosinophilic chronic sinusitis and aspirin-exacerbated eosinophilic chronic sinusitis.

Rhinosinusitis may be further classified as allergic rhinosinusitis and non-allergic rhinosinusitis.

As used herein, the term “allergic rhinosinusitis” refers to rhinosinusitis that occurs in response to exposure to one or more allergens.

As used herein, the term “non-allergic rhinosinusitis” refers to rhinosinusitis caused by, for example, pregnancy, thyroid disorders as a side effect of certain blood pressure and/or topical OTC decongestant medications, structural abnormalities in the nasal septum, structural abnormalities in the nasal filtering structures (turbinates), and/or structural abnormalities in the sinus drainage tracts, nasal polyps and eosinophils. This category also includes vasomotor rhinitis, hormone-induced rhinitis, chemical and irritant-induced rhinitis and rhinitis medicamentosa.

The present disclosure is also enabling for methods of improving one or more parameters associated with inflammation, including inflammation of the nasal mucosa in a subject in need thereof. Thus, in another aspect, there is provided a method for improving one or more parameters associated with inflammation in a subject, the method comprising administering to a subject in need thereof a composition comprising a therapeutically effective combination of (i) cromoglycic acid, or a pharmaceutically-acceptable salt thereof, and (ii) a hyaluronic acid, or a pharmaceutically-acceptable salt thereof (HA).

Illustrative examples of parameters associated with inflammation of the nasal mucosa include the reduction of endoscopic Nasal Polyp Score (NPS). In an embodiment, an NPS of 0 indicates the absence of nasal polyps; an NPS of 1 indicates the presence of small polyps in the middle meatus not reaching below the inferior border of the middle turbinate; an NPS of 2 indicates the presence of multiple polyps occupying the middle meatus; an NPS of 3 indicates large polyps reaching the lower border of the inferior turbinate or polyps medial to the middle turbinate; and an NPS of 4 indicates large polyps causing complete obstruction of the inferior nasal cavity. The maximum score is 8 (i.e., 4 points per nasal cavity). In an embodiment, the method decreases the subject's NPS by about 1 to about 8 points, preferably by 1 point, preferably by 2 points, preferably by 3 points, preferably by 4 points, preferably by 5 points, preferably by 6 points, preferably by 7 points, or more preferably by 8 points. In an embodiment, the method decreases the subject's NPS by about 1 point, or a fraction thereof; preferably by 2 points, or a fraction thereof; preferably by 3 points, or a fraction thereof; preferably by 4 points, or a fraction thereof; preferably by 5 points, or a fraction thereof; preferably by 6 points, or a fraction thereof; preferably by 7 points, or a fraction thereof; or more preferably by 8 points or a fraction thereof. In another embodiment, an NPS of 0 indicates the absence of nasal polyps; an NPS of 1 indicates the presence of small polyps that do not extend beyond the edge of the middle turbinate; an NPS of 2 indicates the presence of polyps that extend beyond the edge of the middle turbinate but do not extend beyond the superior margin of the inferior turbinate; an NPS of 3 indicates larger polyps that extend beyond the superior margin of the inferior turbinate but do not extend beyond the middle of the inferior turbinate; an NPS of 4 indicates larger polyps that extend beyond the middle of the inferior turbinate but do not touch the floor of the nose; and an NPS of 5 indicates large polyps that touch the floor of the nose. In an embodiment, the maximum score is 10 (i.e., 5 points per nasal cavity). In an embodiment, the method decreases the subject's NPS by about 1 to about 10 points, preferably by 1 point, preferably by 2 points, preferably by 3 points, preferably by 4 points, preferably by 5 points, preferably by 6 points, preferably by 7 points, preferably by 8, preferably by 9; or more preferably by 10 points. In an embodiment, the method decreases the subject's NPS by about 1 point, or a fraction thereof; preferably by 2 points, or a fraction thereof; preferably by 3 points, or a fraction thereof; preferably by 4 points, or a fraction thereof; preferably by 5 points, or a fraction thereof; preferably by 6 points, or a fraction thereof; preferably by 7 points, or a fraction thereof; preferably by 8 points or a fraction thereof; preferably by 9 points or a fraction thereof or more preferably by 10 points or a fraction thereof.

The change in NPS may also be represented as a proportion of the subject's NPS prior to treatment. In an embodiment, the method decreases the subject's NPS by about 1%, preferably by about 2%, preferably by about 3%, preferably by about 4%, preferably by about 5%, preferably by about 6%, preferably by about 7%, preferably by about 8%, preferably by about 9%, preferably by about 10%, preferably by about 11%, preferably by about 12%, preferably by about 13%, preferably by about 14%, preferably by about 15%, preferably by about 16%, preferably by about 17%, preferably by about 18%, preferably by about 19%, preferably by about 20%, preferably by about 21%, preferably by about 22%, preferably by about 23%, preferably by about 24%, preferably by about 25%, preferably by about 26%, preferably by about 27%, preferably by about 28%, preferably by about 29%, preferably by about 30, preferably by about 31%, preferably by about 32%, preferably by about 33%, preferably by about 34%, preferably by about 35%, preferably by about 40%, preferably by about 45%, preferably by about 50%, preferably by about 55%, preferably by about 60%, preferably by about 65%, preferably by about 70%, preferably by about 75%, preferably by about 80%, preferably by about 85%, preferably by about 90%, preferably by about 95%, preferably by about 96%, preferably by about 97%, preferably by about 98%, or more preferably by about 99%, or any ranges between these numbers, relative to the subject's NPS prior to treatment.

In an embodiment, a reduction in NPS may correlate with an improvement in one or more other nasal polyp-associated parameters. Such a correlation, however, is not necessarily observed in all cases.

Other parameters associated with inflammation of the nasal mucosa will be familiar to persons skilled in the art, illustrative examples of which include (a) 22-item Sino Nasal Outcome Test (SNOT-22) score; (b) subject-assessed nasal congestion/obstruction, anterior rhinorrhea (runny nose), posterior rhinorrhea (post nasal drip) and loss of sense of smell; (c) number of nocturnal awakenings; (d) Visual Analog Score (VAS) to assess patient-rated rhinosinusitis symptom severity; (e) five-item Asthma Control Questionnaire (ACQ5) score, such as in patients with asthma; (f) Nasal Peak Inspiratory Flow (NPIF); (g) smell test (University of Pennsylvania Smell Identification Test (UPSIT)); (h) physiological parameters, such as measured by nasal endoscopy and CT scan; (i) Lund-Mackay Score; (j) Lund-Kennedy Endoscopy Score (Lund and Kennedy, 1995; Ann Otol Rhinol Laryngol Suppl. 167:17-21); (k) Lund-MacKay Endoscopy Score (Lund and Mackay, 1993; Rhinology. 107:183-184), (l) modified Lund-Kennedy or Lund-MacKay Endoscopy Scores (Lourijsen et al, 2017; Trials, 18-39, (m) Meltzer Clinical Scoring System (Meltzer and Hamilos, 2011; Mayo Clin Proc. 2011; 86(5):427-443), (n) Three Dimensional volumetric measurement of the maxillary sinus and (o) the amount of prostaglandin D₂ (PGD₂) in nasal polyp tissue (see, e.g., US 20100138937).

### 22-Item Sinonasal Outcome Test (SNOT-22) Score

In an embodiment, the methods described herein result in a decrease from baseline of the 22-item Sinonasal Outcome Test (SNOT-22). The SNOT-22 is a questionnaire to assess the impact of chronic rhinosinusitis (CRS) on quality of life. The questionnaire measures items related to sinonasal conditions and surgical treatments. The score ranges from 0 to 110, and higher scores imply greater impact of CRS on Health Related Quality of Life (HRQoL) (see, e.g., Hopkins et al. 2009, Clin. Otolaryngol. 34: 447-454 and Gray et al. 2017, Int Forum Allergy Rhinol. 7(10):945-951).

In an embodiment, the methods disclosed herein result in a decrease in a SNOT-22 score from baseline of at least 1 point at week 4 to week 16 following commencement of treatment. For example, the methods described herein may result in a decrease in a SNOT-22 score at week 4, week 6, week 8, week 12, or week 16 following commencement of treatment. In some embodiments, the methods described herein result in a decrease in a SNOT-22 score from baseline of about 1, preferably about 2, preferably about 3, preferably about 4, preferably about 5, preferably about 6, preferably about 7, preferably about 8, preferably about 9, preferably about 10, preferably about 11, preferably about 12, or more preferably about 13 points or more at week 4, week 6, week 8 or week 12 after commencement of treatment.

### Individual and Total Nasal Symptom Score

Subject-assessed symptoms are assayed by responding to morning and evening individual rhinosinusitis symptom questions using a 0-3 categorical scale (where 0=no symptoms, 1=mild symptoms, 2=moderate symptoms and 3=severe symptoms), and including the symptoms of congestion and/or obstruction, anterior rhinorrhea, posterior rhinorrhea, and loss of sense of smell. Nasal symptoms can be assayed in the day (AM), at night (PM) or both AM and PM.

A loss of sense of smell can also be monitored. The methods described herein can result, for example, in a decrease in loss of sense of smell (i.e., achieving a lower number on the scale) from baseline compared to week 4 to week 16 after the commencement of treatment. For example, a decrease in loss of sense of smell from baseline (e.g., from about 0.5, preferably about 1.0, preferably about 1.5, preferably about 2.0, preferably about 2.5 or more preferably about 3.0 or more) can be detected at week 4, week 6, week 8, week 12, or week 16 following commencement of treatment. In some embodiments, the methods described herein result in a decrease in loss of sense of smell symptom score from baseline by about 0.5, preferably about 1.0, preferably about 1.5, preferably about 2.0, preferably about 2.5 or more preferably about 3.0 or more at week 4, week 8, week 12, or week 16 after the commencement of treatment.

A decrease in congestion and/or obstruction can also be monitored. The methods described herein can result, for example, in a decrease in congestion and/or obstruction (i.e., achieving a lower number on the scale) from baseline compared to week 4 to week 16 after the commencement of treatment. For example, a decrease in congestion and/or obstruction from baseline (e.g., from about 0.5, preferably about 1.0, preferably about 1.5, preferably about 2.0, preferably about 2.5 or more preferably about 3.0 or more) can be detected at week 4, week 6, week 8, week 12, or week 16 after the commencement of treatment. In some embodiments, the methods described herein result in a decrease in congestion and/or obstruction symptom score from baseline by about 0.5, preferably about 1.0, preferably about 1.5, preferably about 2.0, preferably about 2.5 or more preferably about 3.0 or more at week 4, week 8, week 12, or week 16 after the commencement of treatment.

A decrease in runny nose can also be monitored. The methods described herein can result, for example, in a decrease in runny nose (i.e., achieving a lower number on the scale) from baseline compared to week 4 to week 16 following initiation of treatment with the composition described herein. For example, a decrease in runny nose from baseline (e.g., from about 0.5, preferably about 1.0, preferably about 1.5, preferably about 2.0, preferably about 2.5 or more preferably about 3.0 or more) can be detected at week 4, week 6, week 8, week 12, or week 16 after the commencement of treatment. In some embodiments, the methods described herein result in a decrease in runny nose symptom score from baseline by about 0.5, preferably about 1.0, preferably about 1.5, preferably about 2.0, preferably about 2.5 or more preferably about 3.0 or more at week 4, week 8, week 12, or week 16 after the commencement of treatment.

A decrease in post nasal drip can also be monitored. The methods described herein can result, for example, in a decrease in post nasal drip (i.e., achieving a lower number on the scale) from baseline compared to week 4 to week 16 after the commencement of treatment. For example, a decrease in post nasal drip from baseline (e.g., from about 0.5, preferably about 1.0, preferably about 1.5, preferably about 2.0, preferably about 2.5 or more preferably about 3.0 or more) can be detected at week 4, week 6, week 8, week 12, or week 16 after the commencement of treatment. In some embodiments, the methods described herein result in a decrease in post nasal drip symptom score from baseline by about 0.5, preferably about 1.0, preferably about 1.5, preferably about 2.0, preferably about 2.5 or more preferably about 3.0 or more at week 4, week 8, week 12, or week 16 after the commencement of treatment.

A measure of night-time awakenings can also be tracked. For example, a measure of night-time awakenings can be assessed according to the following scores based on subject self-assessment: 0=no symptoms, slept through the night; 1=slept well, but some complaints in the morning; 2=woke up once because of rhinosinusitis symptoms (including early awakening); 3=woke up several times because of symptoms (including early awakening); 4=bad night, awake most of the night because of symptoms. The methods described herein can result, for example, in a decrease in average number of nighttime awakenings per night from baseline of at least about 0.10 times per night at week 4 to week 16 after the commencement of treatment. For example, a decrease in frequency of nighttime awakenings per night from baseline of at least about 0.10 times per night can be detected at week 4, week 6, week 8, week 12, or week 16 after the commencement of treatment. In some embodiments, the methods described herein result in a decrease in average number of nighttime awakenings per night from baseline by about 0.10 times per night, preferably about 0.15 times per night, preferably about 0.20 times per night, preferably about 0.25 times per night, preferably about 0.30 times per night, preferably about 0.35 times per night, preferably about 0.40 times per night, preferably about 0.45 times per night, preferably about 0.50 times per night, preferably about 0.55 times per night, preferably about 0.60 times per night, preferably about 0.65 times per night, preferably about 0.70 times per night, preferably about 0.75 times per night, preferably about 0.80 times per night, preferably about 0.85 times per night, preferably about 0.90 times per night, preferably about 0.95 times per night, preferably about 1.0 time per night, or more preferably about 2.0 times per night or more at week 4, week 8, week 12, or week 16 after the commencement of treatment.

### Visual Analog Score (VAS)

The VAS is a measure to assess patient-related rhinosinusitis symptom severity on a scale of 1 to 10. Mild symptoms are indicated by a score of 0 to 3, moderate symptoms are indicated by a VAS score of >3 to 7, and severe symptoms are indicated by a VAS score of >7 to 10. In some embodiments, the methods described herein result in a decrease in VAS score from baseline of about 0.5 point, preferably about 1 point, preferably about 1.5 points, preferably about 2 points, preferably about 2.5 points, preferably about 3 points, preferably about 3.5 points, or more preferably about 4 points, or more at week 4, week 6 or week 12 after the commencement of treatment. The decrease in VAS score can be detected as early as week 4, and as late as week 12 or later after the commencement of treatment in accordance with the methods described herein.

### 5-Item Asthma Control Questionnaire (ACQ) Score

The ACQ5 measures both the adequacy of asthma control and change in asthma control, which occurs either spontaneously or as a result of treatment. The five questions on the ACQ5 reflect the top-scoring five asthma symptoms: woken at night by symptoms, wake in the mornings with symptoms, limitation of daily activities, shortness of breath and wheeze. Patients respond to the symptom questions on a 7-point scale (0=no impairment, totally controlled; 6=maximum impairment, severely uncontrolled).

In an embodiment, the methods described herein result in a decrease in ACQ5 score from baseline of at least 0.10 point at week 12 following initiation of treatment with the composition described herein. In another embodiment, the methods described herein result in a decrease in ACQ score from baseline of about 0.10 points, preferably about 0.15 points, preferably about 0.20 points, preferably about 0.25 points, preferably about 0.30 points, preferably about 0.35 points, preferably about 0.40 points, preferably about 0.45 points, preferably about 0.50 points, preferably about 0.55 points, preferably about 0.60 points, preferably about 0.65 points, preferably about 0.70 points, preferably about 0.75 points, preferably about 0.80 points, or more preferably about 0.85 points or more at week 4, week 6 or week 12 after the commencement of treatment. The decrease in ACQ score may be detected as early as week 4, and as late as week 12 or later after the commencement of treatment in accordance with the methods described herein.

### Nasal Peak Inspiratory Flow (NPIF)

The Nasal Peak Inspiratory Flow (NPIF) represents a physiologic measure of air flow through both nasal cavities during forced inspiration and/or expiration expressed in liters per minute. Nasal inspiration correlates most with the subjective feeling of obstruction and is used to monitor nasal flow. In an embodiment, the methods described herein result in an increase in NPIF from baseline by about 0.10 liters per minute, preferably by about 0.15 liters per minute, preferably by about 0.20 liters per minute, preferably by about 0.25 liters per minute, preferably by about 0.30 liters per minute, preferably by about 0.35 liters per minute, preferably by about 0.40 liters per minute, preferably by about 0.45 liters per minute, preferably by about 0.50 liters per minute, preferably by about 0.55 liters per minute, preferably by about 0.60 liters per minute, preferably by about 0.65 liters per minute, preferably by about 0.70 liters per minute, preferably by about 0.75 liters per minute, preferably by about 0.80 liters per minute, or more preferably by about 0.85 liters per minute, or more at week 4, week 6 or week 12 after the commencement of treatment. The increase in NPIF score can be detected as early as week 4, and as late as week 12 or later after the commencement of treatment in accordance with the methods described herein.

### University of Pennsylvania Smell Identification Test (UPSIT)

The UPSIT is a method to quantitatively assess human olfactory function. The test consists of samples of odorants, and the subject has to describe the odor. The score is based on the number of correct answers. This test can distinguish patients with a normal sense of smell (“normosmia”) from those with different levels of reduction (“mild, moderate and severe microsmia”) or loss (“anosmia”). In an embodiment, the methods described herein result in an increase in UPSIT score from baseline by about 0.5 points, preferably about 1 point, preferably about 1.5 points, preferably about 2 points, preferably about 2.5 points, preferably about 3 points, or more preferably about 3.5 points or more at week 4, week 6 or week 12 after the commencement of treatment. The increase in UPSIT score may be detected as early as week 4, and as late as week 12 or later after the commencement of treatment in accordance with the methods described herein.

### Physiological Parameters

The efficacy of the compositions and methods described herein can also be determined by measuring the effect of physiological parameters, such as within the nasal cavities, e.g., by nasal endoscopy or computed tomography (CT) scan.

### Lund-Mackay Score

The Lund-Mackay scoring system is based on localization with points given for degree of opacification: 0=normal, 1=partial opacification, 2=total opacification. These points are then applied to the maxillary, anterior ethmoid, posterior ethmoid, sphenoid, frontal sinus and osteomeatal complex on each side, deriving a maximum score of 12 per side. For patients in whom the osteomeatal complex (OC) is missing (because of a previous surgery) the location of the former OC is considered and a score is provided, as if the OC was there. In an embodiment, the methods described herein result in a decrease in Lund-Mackay score from baseline by about 0.10 points, preferably about 0.15 points, preferably about 0.20 points, preferably about 0.25 points, preferably about 0.30 points, preferably about 0.35 points, preferably about 0.40 points, preferably about 0.45 points, preferably about 0.50 points, preferably about 0.55 points, preferably about 0.60 points, preferably about 0.65 points, preferably about 0.70 points, preferably about 0.75 points, preferably about 0.80 points, or more preferably about 0.85 points or more at week 4, week 6 or week 12 after the commencement of treatment. The decrease in Lund-Mackay score may be detected as early as week 4, and as late as week 12 or later after the commencement of treatment in accordance with the methods described herein.

### Lund-Kennedy and Modified Lund-Kennedy Score

The Lund-Kennedy and Modified Lund-Kennedy endoscopic scoring systems are described in Abhishek K R. (2018, Exp Rhinol Otolaryngol. 2(4). ERO.000541) and Psaltis et al. (2014, Laryngoscope, 124(10):2216-2223). The decrease in Modified Lund-Kennedy score may be detected as early as week 4, and as late as week 12 or later after the commencement of treatment in accordance with the methods described herein.

### Three-Dimensional Volumetric Measurement of Maxillary Sinus

This value is used to calculate the volume of air (mL); the volume of mucosa (mL); the percent sinus occupied by disease; and the thickness of lateral wall in the maxillary sinus. In an embodiment, the methods described herein result in an increase in the Three-Dimensional volumetric measurement.

### Quality of Life (QoL) Questionnaires

Various QoL Questionnaires can also be used to monitor the efficacy of treatment, illustrative examples of which include the Short-Form-36 (SF-36) Questionnaire, the Eurogol-5D (EQ-5D), the nasal polyp related resource use questionnaire, and the patient qualitative self-assessment.

The SF-36 is a 36-item questionnaire that measures eight multi-item dimensions of health: physical functioning (10 items) social functioning (2 items) role limitations due to physical problems (4 items), role limitations due to emotional problems (3 items), mental health (5 items), energy/vitality (4 items), pain (2 items), and general health perception (5 items). For each dimension, item scores are coded, summed, and transformed on a scale from 0 (worst possible health state measured by the questionnaire) to 100 (best possible health state). Two standardized summary scores can also be calculated from the SF-36; the physical component summary (PCS) and the mental health component summary (MCS).

The EQ-5D is a standardized health-related quality of life questionnaire developed by the EuroQoI Group in order to provide a simple, generic measure of health for clinical and economic appraisal and inter-disease comparisons. EQ-5D, designed for self-completion by patients, consists of two parts, the EQ-5D descriptive system and the EQ VAS. The EQ-5D descriptive system comprises 5 dimensions: mobility, self-care, usual activities, pain/discomfort and anxiety/depression; and each dimension has 3 levels: no problem, some problems, severe problems. The EQ Visual Analogue Scale (VAS) records the respondent's self-rated health on a vertical visual analogue scale. The EQ VAS ‘thermometer’ has endpoints of 100 (Best imaginable health state) at the top and 0 (Worst imaginable health state) at the bottom.

The nasal polyp related resource use questionnaire is a questionnaire of health care resource utilization for nasal polyposis, including specialist visits, emergency care visits, sick leaves, days off etc.

### Nasal Polyp-Associated Biomarkers

Illustrative examples of suitable nasal polyp-associated biomarkers include any one or more of IgE (total IgE or antigen-specific IgE), thymus and activation regulated chemokine (TARC, also known as CCL17), eotaxin-3 and ECP. In an embodiment, the nasal polyp-associated biomarker is detected from a biological sample derived from a subject, wherein the biological sample is selected from the group consisting of cultures, cells, tissues, blood, saliva, nasal secretions, cerebrospinal fluid, pleural fluid, milk, lymph, sputum, semen, needle aspirates, and the like. Biological samples may be obtained using methods known to persons skilled in the art, non-limiting examples of which include nasal secretion samples obtained from smears, blown secretions, imprints, lavage, swabs, brushes and the like.

A normal IgE level in healthy subjects is less than about 100 kU/L. Thus, in an embodiment, the methods described herein result in a decrease in an elevated serum IgE level in the subject, wherein the elevated serum IgE level is greater than about 100 kU/L, preferably greater than about 150 kU/L, preferably greater than about 500 kU/L, preferably greater than about 1000 kU/L, preferably greater than about 1500 kU/L, preferably greater than about 2000 kU/L, preferably greater than about 2500 kU/L, preferably greater than about 3000 kU/L, preferably greater than about 3500 kU/L, preferably greater than about 4000 kU/L, preferably greater than about 4500 kU/L, or more preferably greater than about 5000 kU/L. Also disclosed herewith are methods by which serum levels of antigen-specific IgE levels are determined.

TARC levels in healthy subjects are in the range of 106 ng/L to 431 ng/L, with a mean of about 239 ng/L. Thus, in an embodiment, the methods described herein result in a decrease in an elevated serum TARC level in the subject, wherein the elevated serum TARC level is greater than about 431 ng/L, preferably greater than about 500 ng/L, greater than about 1000 ng/L, preferably greater than about 1500 ng/L, preferably greater than about 2000 ng/L, preferably greater than about 2500 ng/L, preferably greater than about 3000 ng/L, preferably greater than about 3500 ng/L, preferably greater than about 4000 ng/L, preferably greater than about 4500 ng/L, or more preferably greater than about 5000 ng/L.

Improvement of a nasal polyp-associated parameter, such as a nasal polyp-associated parameter described above, can also be expressed as a percentage. For example, a score can be improved by 30% or more, preferably by 40% or more, preferably by 50% or more, preferably by 60% or more, preferably by 70% or more, or more preferably by 80% or more in the subject by the methods described herein.

Biomarker expression, referred to elsewhere herein, may be assayed by detection of protein or RNA in serum. In some embodiments, RNA samples are used to determine RNA levels (non-genetic analysis), e.g., RNA levels of biomarkers; and in other embodiments, RNA samples are used for transcriptome sequencing (e.g., genetic analysis). In an embodiment, the biomarker is circular RNA. Suitable methods for detecting circular RNA will be familiar to persons skilled in the art, illustrative examples of which are described in Jeck and Sharpless (Nature Biotechnology, 2014, 32(5):453-461), the contents of which are incorporated herein by reference.

An “improvement in a parameter associated with inflammation of the nasal mucosa”, as used herein, typically means an increase from baseline of one or more of NPIF, UPSIT, and/or a decrease from baseline of one or more of SNOT-22 score, subject-assessed nasal congestion/obstruction, anterior rhinorrhea (runny nose), posterior rhinorrhea (post nasal drip) and loss of sense of smell; number of nocturnal awakenings; VAS score; Lund-Mackay score; and 3D volumetric scores; and ACQ5 score in patients with asthma. In the context of a parameter associated with inflammation of the nasal mucosa, the term “baseline” is used herein to mean the numerical value of the parameter for a subject prior to or at the time of commencement of treatment in accordance with the methods described herein.

To determine whether a parameter associated with inflammation of the nasal mucosa has improved (whether increased or decreased), the parameter is typically quantified at baseline and at a time point after the commencement of treatment in accordance with the methods described herein. For example, a parameter associated with inflammation of the nasal mucosa may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the commencement of treatment in accordance with the methods described herein. The parameter may be measured daily (e.g., once or twice per day), weekly, biweekly, or monthly. The parameter may also be measured daily and the mean value determined over the course of a month is compared to baseline.

### Combination Therapies

The methods and uses described herein may suitably be combined with one or more additional treatment modalities, including for the treatment of a condition associated with inflammation and/or for promoting wound healing, illustrative examples of which will be familiar to persons skilled in the art. In an embodiment, the composition described herein is formulated for co-administration, or administration in combination, with an additional therapeutic agent. As used herein, the expression “in combination with” and “co-administration” mean that the additional therapeutic agent is administered before, after, or concurrent with the composition described herein. For example, when administered “before” the composition described herein, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the composition described herein. When administered “after” the composition described herein, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the composition described herein. Administration “concurrent” with the composition described herein typically means that the additional therapeutic agent is administered to the subject in a separate dosage form within less than 5 minutes (before, after, or at the same time) of administration of the composition described herein, or administered to the subject as a single combined dosage formulation comprising both the additional therapeutic agent and composition described herein.

Suitable additional therapeutic agents will be familiar to persons skilled in the art, illustrative examples of which include antibiotics (e.g., macrolides) and steroids (e.g., prednisolone). In an embodiment, the additional therapeutic agent is selected from the group consisting of an antibiotic and a steroid. In an embodiment, the additional therapeutic agent is a steroid. In an embodiment, the steroid is a corticosteroid. In an embodiment, the corticosteroid is prednisolone. In an embodiment, the additional therapeutic agent is an antibiotic. Suitable antibiotics will be familiar to persons skilled in the art, illustrative examples of which include macrolides (e.g., azithromycin (0.5%, 1%, and 1.5%), clarithromycin (1%), erythromycin (2%)), mupirocin (2%) and aminoglycoside (e.g., Tobramycin, 0.3%). In an embodiment, the additional therapeutic agent is an antimicrobial and/or antifungal agent. An illustrative example includes clioquinol (1-3% preparation).

In an embodiment, the additional therapeutic agent is an antimicrobial peptide. Suitable antimicrobial peptides will be familiar to persons skilled in the art, illustrative examples of which are described elsewhere herein. Other illustrative examples of suitable additional therapeutic agents include sulfasalazine (mesalazine, an aminosalicylate anti-inflammatory+sulfapyridine, a sulfanilamide antibacterial), antihistamines, leukotriene inhibitors (e.g., montelukast) and verapamil (2.5 mg in 1 ml in 200 ml saline).

In certain embodiments, multiple doses of the composition described herein may be administered to a subject over a defined time course. Illustrative examples include sequentially administering to a subject multiple doses of the composition described herein. As used herein, “sequentially administering” means that each dose of the composition described herein is administered to the subject at a different point in time (such as on different days separated by a predetermined interval, e.g., hours, days, weeks or months). The methods described herein may also comprise sequentially administering to the patient a single initial dose of the composition described herein, followed by one or more secondary doses of the composition described herein, and optionally followed by one or more tertiary doses of the composition described herein. The terms “initial dose,” “secondary doses,” “tertiary doses” and so on refer to the temporal sequence of administration of the composition described herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses, and so on. The initial, secondary, tertiary and any subsequent doses may all contain the same amount of the cromoglycic acid or salt thereof, but will generally differ from one another in terms of frequency of administration. In other embodiments, the amount of the cromoglycic acid or salt thereof in the initial, secondary, tertiary and/or any subsequent doses will vary from one another (e.g., adjusted up or down as appropriate) during the course of treatment.

In an embodiment, each secondary, tertiary and/or any subsequent dose is administered 1 to 14 (e.g., 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or more) weeks after the immediately preceding dose. The phrase “the immediately preceding dose,” as used herein, means, in a sequence of multiple administrations, the dose of the cromoglycic acid or salt thereof that is administered to a subject prior to the administration of the very next dose in the sequence with no intervening doses.

The methods described herein may also include administering to a subject any number of secondary, tertiary and/or any subsequent doses of the composition described herein. In an embodiment, only a single secondary dose is administered to the subject. In another embodiment, two or more (e.g., 2, 3, 4, 5, 6, 7, 8, or more) secondary doses are administered to the patient. Similarly, in some embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the subject 1 to 2 weeks after the immediately preceding dose. Similarly, in embodiments involving multiple tertiary doses, each tertiary dose may be administered at the same frequency as the other tertiary doses. For example, each tertiary dose may be administered to the subject 2 to 4 weeks after the immediately preceding dose. Alternatively, the frequency at which the secondary and/or tertiary doses are administered to a subject can vary over the course of the treatment regimen. The frequency of administration may also be adjusted during the course of treatment by a physician depending on the needs of the individual patient following clinical examination.

In certain embodiments, the initial dose (e.g., a “loading dose”) is higher than either the secondary, tertiary or any subsequent doses. For example, the initial dose can be a loading dose, which is 1.5 times, 2 times, 2.5 times, 3 times or more greater than the secondary dose.

### Subject

As used herein, “subject” and “subject in need thereof” mean a human or non-human animal. The subject may exhibit one or more symptoms or conditions associated with inflammation, including inflammation of the nasal mucosa. In an embodiment, the subject has been diagnosed with one of chronic rhinosinusitis with nasal polyps or chronic rhinosinusitis without nasal polyps. In an embodiment, the subject has bilateral nasal polyps, and a nasal polyp score of at least 4 out of a maximum of 8 for both nostrils, with at least a score of 2 for each nostril. In another embodiment, the polyps are in the middle meatus. In an embodiment, the presence of nasal polyps is confirmed by endoscopy. In other embodiments, the subject has bilateral mucosal disease, which may be confirmed by a method such as CT scan. As used herein “bilateral mucosal disease” is an inflammation of the mucous lining of the sinus cavities, e.g., the maxillary sinus cavities. In some embodiments, bilateral nasal polyposis (e.g., a nasal polyp score of at least 4 out of a maximum of 8 for both nostrils, with at least a score of 2 for each nostril) persists even after a treatment regimen of inhaled corticosteroids (INCS), such as where the INCS was administered for at least 6 weeks, at least 7 weeks, at least 8 weeks, or longer.

In an embodiment, the subject has anterior and/or posterior mucopurulent drainage, nasal obstruction, and a decreased sense of smell. In an embodiment, the subject has had symptoms of chronic rhinosinusitis with nasal polyps for 6 weeks, preferably for 7 weeks, preferably for 8 weeks, preferably for 9 weeks, preferably for 10 weeks, preferably for 11 weeks, or more preferably for 12 weeks or more. In another embodiment, the subject has received a previous treatment, such as with an intranasal corticosteroid (e.g., MFNS), for at least 4 weeks, preferably for at least 5 weeks, preferably for at least 6 weeks, preferably for at least 7 weeks, preferably for at least 8 weeks, preferably for at least 9 weeks, or more preferably for at least 10 weeks or more, prior to the commencement of treatment. In another embodiment, the subject will continue to receive INCS while receiving treatment in accordance with the methods disclosed herein. In another embodiment, the subject stops receiving INCS before receiving treatment in accordance with the methods disclosed herein, or the subject stops receiving treatment with INCS if treatment in accordance with the methods disclosed herein is effective to treat the nasal polyps. In an embodiment, the dose of the INCS is tapered before stopping treatment completely.

A subject in need thereof may further have been diagnosed with nasal polyposis or a condition associated with nasal polyposis, such as chronic rhinosinusitis with nasal polyps, allergic or non-allergic rhinitis, or allergic or non-allergic rhinosinusitis. The diagnosis may be on the basis of one or more of the following: (a) 22-item Sino Nasal Outcome Test (SNOT-22) score; (b) subject-assessed nasal congestion/obstruction, anterior rhinorrhea, posterior rhinorrhoea and loss of sense of smell; (c) number of nocturnal awakenings; (d) Visual Analog Score (VAS) to assess patient-rated rhinosinusitis symptom severity; (e) five-item Asthma Control Questionnaire (ACQ5) score in patients with asthma; (f) Nasal Polyp Score (NPS); (g) Nasal Peak Inspiratory Flow (NPIF); (h) smell test (University of Pennsylvania Smell Identification Test (UPSIT); (i) physiological parameters, such as measured by nasal endoscopy and CT scan; j) Lund-Mackay Score; (k) Three Dimensional volumetric measurement of the maxillary sinus, (l) Lund-Kennedy Score and (m) Modified Lund-Kennedy Score.

In an embodiment, a subject is a human patient with chronic symptoms of sinusitis, which are the presence of at least two of the following symptoms: nasal blockade/obstruction/congestion or nasal discharge (anterior/posterior nasal drip), facial pain/pressure and reduction or loss of smell.

In an embodiment, the subject is a human patient with a SNOT-22 score of greater than about 7, preferably greater than about 10, preferably greater than about 15, preferably greater than about 20, preferably greater than about 25, preferably greater than about 30, preferably greater than about 35, preferably greater than about 40, preferably greater than about 45, or more preferably greater than about 50. In another embodiment, the subject is a human patient who exhibits a Lund-Mackay score of greater than about 4, preferably greater than about 5, preferably greater than about 6, preferably greater than about 7, preferably greater than about 8, preferably greater than about 9, preferably greater than about 10, preferably greater than about 11, preferably greater than about 12, or more preferably greater than about 13.

In another embodiment, the subject, prior to receiving treatment in accordance with the methods described herein, has been prescribed or is currently receiving additional medication (i.e., background therapy). Background therapy can be, for example, an intranasal corticosteroid (INCS, or ICS), such as Mometasone furoate nasal spray (MFNS; Nasonex®). In another embodiment, the subject is an asthma patient who, prior to receiving treatment in accordance with the methods described herein, has been prescribed or is currently receiving INCS in combination with a long-acting beta₂ adronergic antagonist (LABA). Illustrative examples of suitable INCS/LABA therapies include fluticasone/salmeterol combination therapy and budesonide/formoterol combination therapy. In some embodiments, the background therapy is selected from the group consisting of a nasal saline, a topical decongestant, a topical anaesthetic, a leukotriene antagonist and a systemic antihistamine. In an embodiment, the subject continues the background therapy after commencement of treatment in accordance with the methods described herein. In other embodiments, the subject stops receiving background therapy (e.g., at once or gradually) before commencement of treatment in accordance with the methods described herein.

The difference between the value of the parameter at a particular time point following initiation of treatment and the value of the parameter at baseline is used to establish whether there has been an “improvement” in the nasal associated parameter (e.g., an increase or decrease, as the case may be, depending on the specific parameter being measured).

The invention will now be described with reference to the following Examples which illustrate some preferred aspects of the present invention. However, it is to be understood that the particularity of the following description of the invention is not to supersede the generality of the preceding description of the invention.

### Example

### Example 1: Intranasal Administration of SCG and Hyaluronic Acid Inhibits Recurrence of Nasal Polyps and Promotes Wound Healing Following Endoscopic Sinus Surgery

A topical intranasal composition comprising sodium cromoglycate (SCG) and hyaluronic acid (HA) was prepared by mixing 0.4 g powdered SCG with 10 ml of hyaluronic acid (PureRegen™; BioRegen Biomedical, China; final concentration of SCG is 4% weight/volume). SCG powder was added to PureRegen™ in a sterile container and mixed together.

All patients had nasal polyposis with symmetric pan-sinus disease on imaging and clinical examination. No patients had previous surgery. All patients had failed to respond to conventional therapy with intranasal corticosteroids. All patients gave informed consent for trial of application of sodium cromoglycate/hyaluronic acid gel. Endoscopic sinus surgery was undertaken to clear nasal polyps and open all paranasal sinuses on both sides. Patient 1 had treatment gel (SCG+HA) applied to both sinus cavities at end of surgical procedure. Patients 2 through 9 had treatment gel (SCG+HA) applied to one sinus cavity, and standard hyaluronic acid gel (PureRegen™ only) was applied to the contralateral sinus cavity (control). Sinus cavities were inspected with a 2.9 mm, 30 degree rigid sinoscope at each post-operative appointment, and treatment and control sinus cavities were scored using the following modified Lund Kennedy Sinus Endoscopy Scoring System, as described in Table 1 below:

TABLE 1
Modified Lund Kennedy Sinus Endoscopy Scoring System

| Polyp: | Oedema: | Discharge: | Crusting: |
|---|---|---|---|
| 0 = absence of polyp | 0 = normal | 0 = none | 0 = no crust |
| 1 = polyp confined | 1 = non-polypoid | 1 = thin clear | 1 = mild, |
| to middle meatus | oedema | mucus | patchy over |
| | | | edges |
| 2 = polyp beyond | 2 = polypoid | 2 = thick, | 2 = severe, |
| middle meatus | oedema | tenacious or | overlying |
| | | purulent mucus | mucosa |

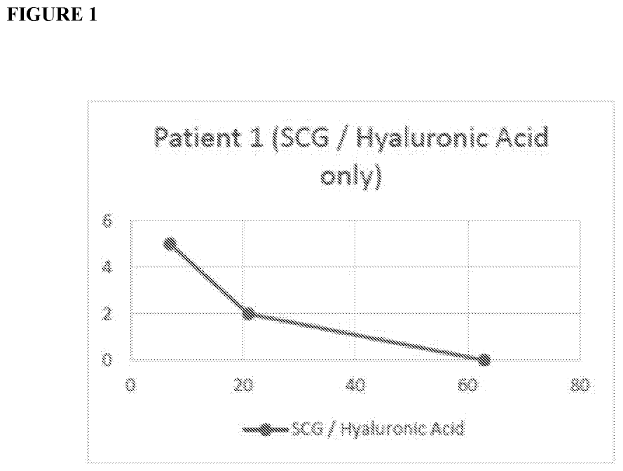

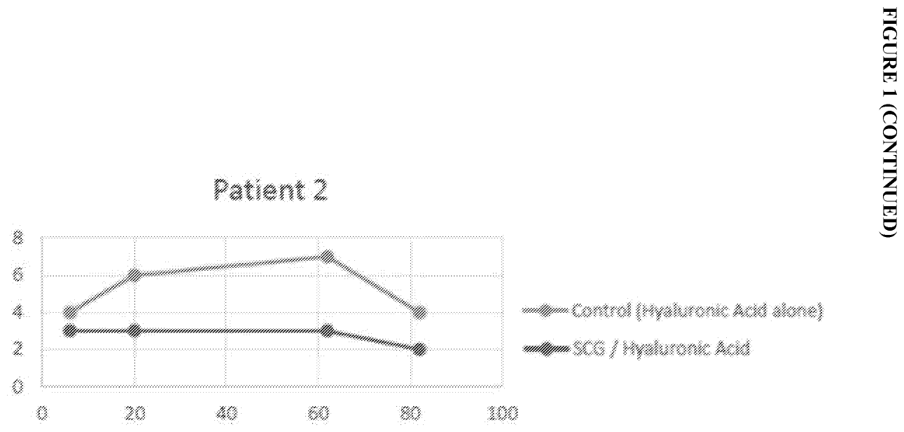

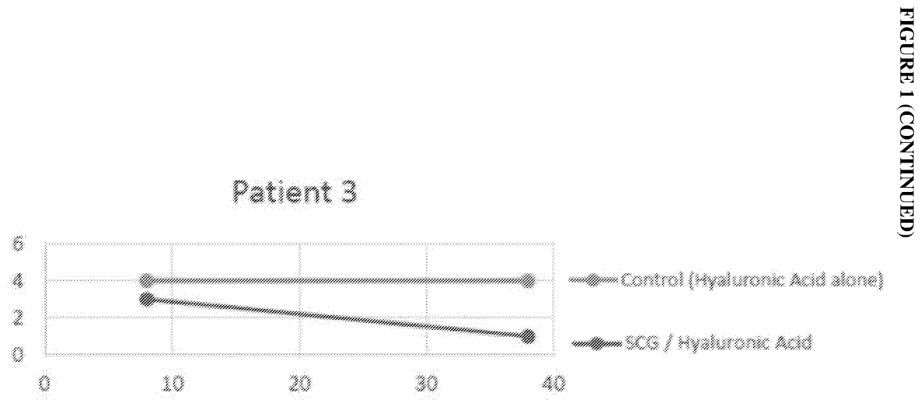

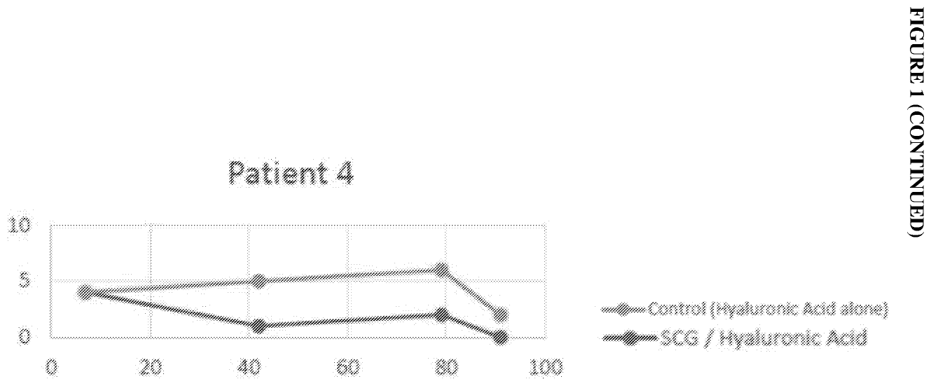

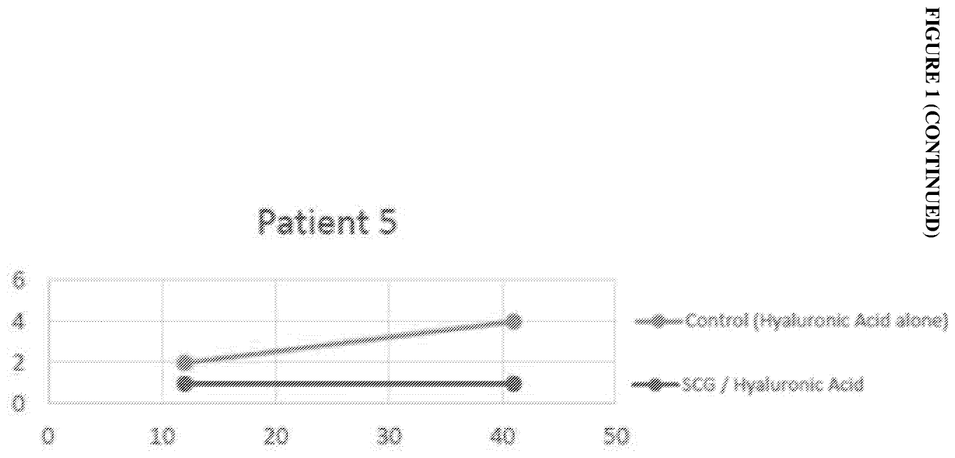

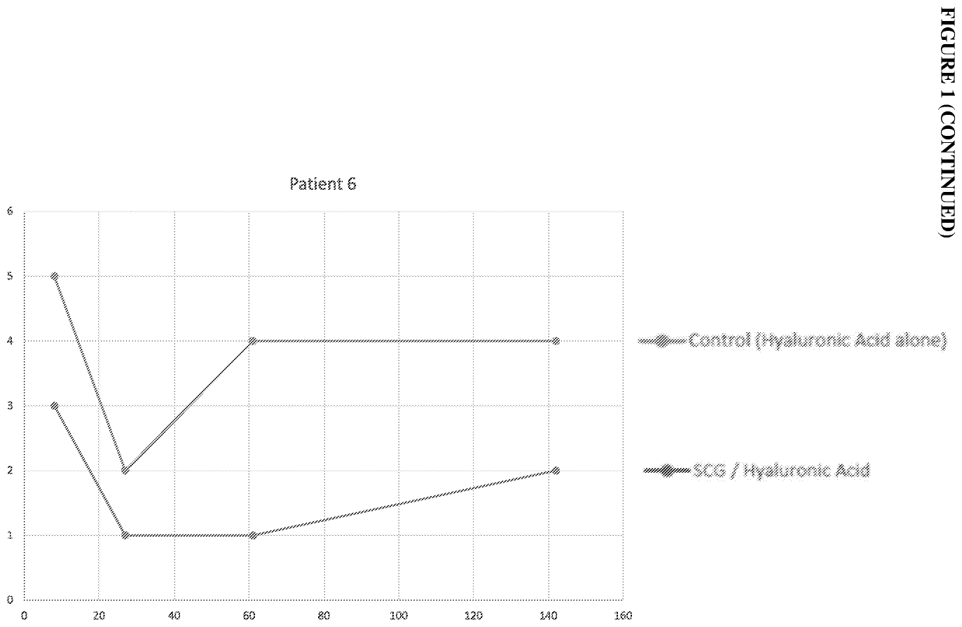

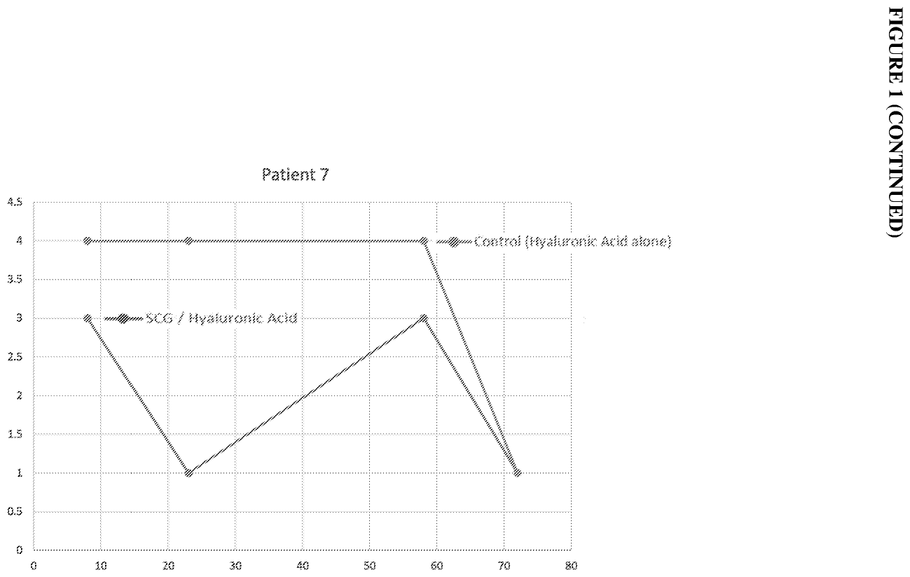

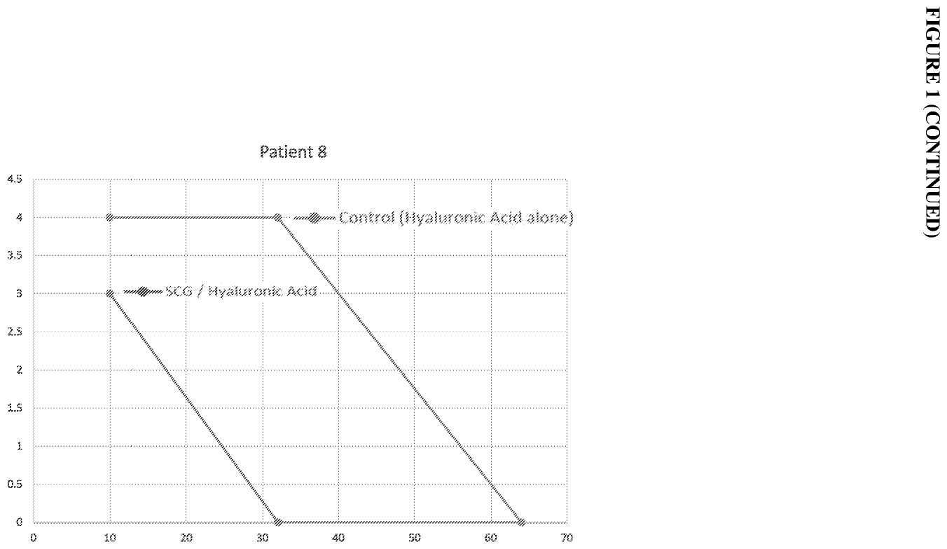

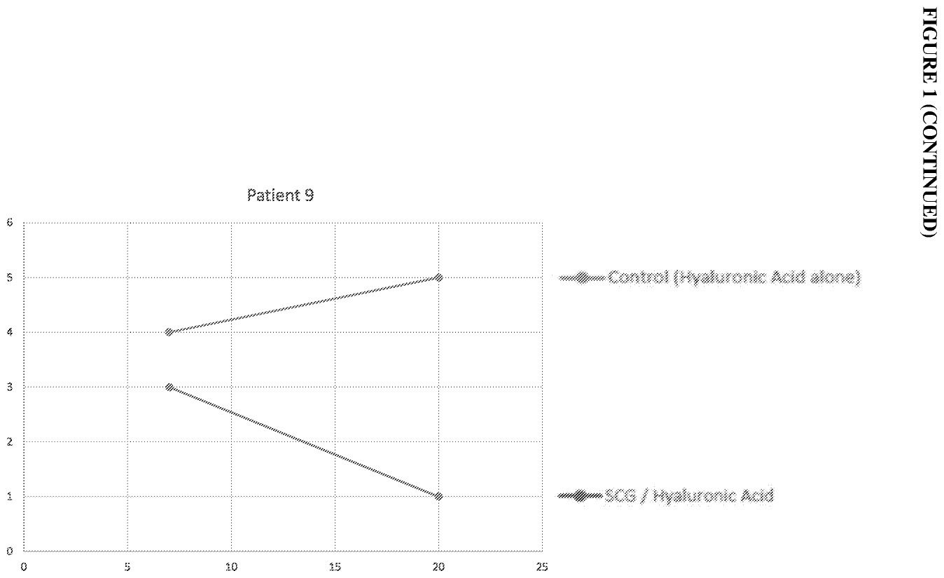

As set in Tables 2 and 3, below, in all cases, the sinus cavity that received treatment with SCG+HA showed more rapid and improved wound healing when compared to the contralateral sinus cavity. This effect appeared to persist beyond 90 days (see also FIG. 1).

TABLE 2
Results
PATIENT DATA -SODIUM CROMOGLYCATE/HYALURONIC ACID SINUS GEL

| | | Post | | Polyp | Oedema | Discharge | Crust | Modified | Modified |
|---|---|---|---|---|---|---|---|---|---|
| | Surgery | Op | | (Rx/ | (Rx/ | (Rx/ | (Rx/ | LKS | LKS |
| Patient | Date | Days | Comments | Control) | Control) | Control) | Control) | Rx | Control |
| Patient 1 | 3 Sep. 2020 | 7 | Patchy healing <50% | 0/— | 2/— | 2/— | 1/— | 5 | |
| (Both sides | | | | | | | | | |
| treatment | | | | | | | | | |
| only) | | | | | | | | | |
| | | 21 | >50% healed | 0/— | 1/— | 1/— | 0/— | 2 | |
| | | 63 | All healed | 0/— | 0/— | 0/— | 0/— | 0 | |
| Patient 2 | 3 Sep. 2020 | 6 | All areas oedematous | 0/0 | 1/2 | 1/1 | 1/1 | 3 | 4 |
| | | 20 | All areas oedematous, | 0/0 | 1/2 | 1/2 | 1/2 | 3 | 6 |
| | | | Control side polypoid | | | | | | |
| | | 62 | | 0/1 | 1/1 | 1/2 | 1/2 | 3 | 7 |
| | | 82 | Patchy healing <50% | 0/0 | 1/2 | 1/2 | 0/0 | 2 | 4 |
| Patient 3 | 7 Sep. 2020 | 8 | Patchy healing <50% | 0/0 | 1/2 | 1/1 | 1/1 | 3 | 4 |
| | | 38 | >50% healed | 0/0 | 1/2 | 0/1 | 0/1 | 1 | 4 |
| Patient 4 | 22 Sep. 2020 | 7 | Patchy healing <50% | 0/0 | 2/2 | 1/1 | 1/1 | 4 | 4 |
| | | 42 | Patchy healing <50% | 0/0 | 0/2 | 0/2 | 1/1 | 1 | 5 |
| | | | (control); >50% healed | | | | | | |
| | | | (Rx) | | | | | | |
| | | 79 | | 0/1 | 1/2 | 1/2 | 0/1 | 2 | 6 |
| | | 91 | | 0/0 | 0/1 | 0/1 | 0/0 | 0 | 2 |
| Patient 5 | 29 Oct. 2020 | 12 | >50% healed | 0/0 | 1/1 | 0/0 | 0/1 | 1 | 2 |
| | | 41 | | 0/0 | 1/2 | 0/2 | 0/0 | 1 | 4 |

TABLE 3
Results continued
PATIENT DATA - NOVEL SODIUM CROMOGLYCATE/HYALURONIC ACID SINUS GEL

| | | Post | | Polyp | Oedema | Discharge | Crust | Modified | Modified |
|---|---|---|---|---|---|---|---|---|---|
| | Surgery | Op | | (Rx/ | (Rx/ | (Rx/ | (Rx/ | LKS | LKS |
| Patient | Date | Days | Comments | Control) | Control) | Control) | Control) | Rx | Control |
| Patient 6 | | 8 | Patchy healing <50% | 0/0 | 1/2 | 1/2 | 1/1 | 3 | 5 |
| | | 27 | >50% healed | 0/0 | 1/1 | 0/1 | 0/0 | 1 | 2 |
| | | 61 | | 0/0 | 1/2 | 0/2 | 0/0 | 1 | 4 |
| | | 142 | | 0/1 | 1/2 | 1/1 | 0/0 | 2 | 4 |
| Patient 7 | | 8 | Patchy healing | 0/0 | 1/2 | 1/1 | 1/1 | 3 | 4 |
| | | | Tx side | | | | | | |
| | | 23 | | 0/0 | 0/2 | 1/2 | 0/0 | 1 | 4 |
| | | 58 | | 0/1 | 1/1 | 1/2 | 1/1 | 3 | 4 |
| | | 72 | | 0/0 | 1/1 | 0/0 | 0/0 | 1 | 1 |
| Patient 8 | | 10 | Patchy healing <50% | 0/0 | 1/2 | 1/1 | 1/1 | 3 | 4 |
| | | 32 | >50% healed | 0/0 | 0/2 | 0/1 | 0/1 | 0 | 4 |
| | | 64 | | 0/0 | 0/0 | 0/0/ | 0/0 | 0 | 0 |
| Patient 9 | | 7 | Patchy healing <50% | 0/0 | 1/2 | 1/1 | 1/1 | 3 | 4 |
| | | 20 | | 0/0 | 0/2 | 0/2 | 1/1 | 1 | 5 |

These data show that SCG in combination with HA is a useful therapeutic composition for reducing inflammation, promoting wound healing and suppressing nasal polyp recurrence in the sinus mucosa.

1. A topical composition comprising a therapeutically effective combination of (i) cromoglycic acid, or a pharmaceutically-acceptable salt thereof, and (ii) hyaluronic acid, or a pharmaceutically-acceptable salt thereof (HA).

2. The composition of claim 1, wherein the cromoglycic acid, or the pharmaceutically-acceptable salt thereof, is present in an amount of from about 0.5% to about 10% (w/v).

3. The composition of claim 2, wherein the cromoglycic acid, or the pharmaceutically-acceptable salt thereof, is present in an amount of from about 1% to about 5% (w/v).

4. The composition of claim 2, wherein the cromoglycic acid, or the pharmaceutically-acceptable salt thereof, is present in an amount of from about 3% to about 5% (w/v).

5. The composition of claim 2, wherein the cromoglycic acid, or the pharmaceutically-acceptable salt thereof, is present in an amount of from about 4% (w/v).

6. The composition of any one of claims 1 to 5, wherein the pharmaceutically-acceptable salt of cromoglycic acid is sodium cromoglycate.

7. The composition of any one of claims 1 to 6, wherein the HA has an average molecular weight of from about 5 kDa to about 20,000 kDa.

8. The composition of claim 7, wherein the HA has an average molecular weight of from about 50 kDa to about 8,000 kDa.

9. The composition of claim 7, wherein the HA has an average molecular weight of from about 1000 kDa to about 8,000 kDa.

10. The composition of any one of claims 1 to 9, wherein the HA has a viscosity of from about 1,000 mPa/s to about 200,000 mPa/s.

11. The composition of claim 10, wherein the HA has a viscosity of from about 10,000 mPa/s to about 200,000 mPa/s.

12. The composition of claim 10, wherein the HA has a viscosity of from about 100,000 mPa/s to about 200,000 mPa/s.

13. The composition of any one of claims 1 to 12, wherein the HA comprises cross-linked hyaluronans.

14. The composition of any one of claims 1 to 13, wherein the HA is a water-insoluble gel.

15. The composition of any one of claims 1 to 14, wherein the composition is formulated for intra-operative administration.

16. The composition of any one of claims 1 to 15, wherein the composition is an intranasal composition.

17. A device configured for the delivery of the composition of any one of claims 1 to 16.

18. The device of claim 17, for the intranasal delivery of the composition.

19. The device of claim 17 or claim 18, wherein the device is an intranasal device.

20. The device of any one of claims 17 to 19, wherein the device is a stent.

21. The device of any one of claims 17 to 20, wherein the device comprises the composition of any one of claims 1 to 15.

22. The composition of any one of claims 1 to 16 or the device of any one of claims 17 to 21, wherein the composition is formulated, and the device is configured, for co-administration with an additional therapeutic agent.

23. The composition or device of 22, wherein the additional therapeutic agent is selected from the group consisting of a steroid, an antibiotic and an antimicrobial peptide (AMP) or a fusion protein comprising same.

24. A method of treating a condition associated with inflammation, the method comprising administering to a subject in need thereof the composition of any one of claims 1 to 16 or the device of any one of claims 17 to 21.

25. The method of claim 24, wherein the condition is associated with inflammation of the nasal mucosa.

26. The method of claim 25, wherein the condition is associated with inflammation of the sinonasal mucosa.

27. The method of claim 25 or claim 26, wherein the condition is chronic rhinosinusitis (CRS).

28. The method of claim 27, wherein the CRS is chronic rhinosinusitis with nasal polyps (CRSwNP) or chronic rhinosinusitis without nasal polyps (CRSsNP).

29. The method of claim 28, wherein the CRS is chronic rhinosinusitis with nasal polyps (CRSwNP).

30. The method of claim 29, wherein the composition or device is administered intraoperatively following endoscopic sinus surgery.

31. The method of any one of claims 25 to 30, wherein the composition or device reduces inflammation of the nasal mucosa as determined by Lund Kennedy Sinus Endoscopy Score when compared to a composition or device comprising the HA in the absence of the cromoglycic acid, or the pharmaceutically-acceptable salt thereof.

32. The method of claim 31, wherein the reduction in inflammation of the nasal mucosa, as determined by Lund Kennedy Sinus Endoscopy Score, is evident by at least about 20 days following administration of the composition or device.

33. The method of claim 31, wherein the reduction in inflammation of the nasal mucosa, as determined by Lund Kennedy Sinus Endoscopy Score, is evident by at least about 40 days following administration of the composition or device.

34. The method of any one of claims 31 to 33, wherein the reduction in inflammation of the nasal mucosa, as determined by Lund Kennedy Sinus Endoscopy Score, persists for at least about 60 days following administration of the composition or device.

35. The method of claim 34, wherein the reduction in inflammation of the nasal mucosa, as determined by Lund Kennedy Sinus Endoscopy Score, persists for at least about 80 days following administration of the composition or device.

36. A method of promoting wound healing, the method comprising administering to a subject in need thereof the composition of any one of claims 1 to 16 or the device of any one of claims 17 to 21.

37. The method of claim 36 for promoting wound healing of the nasal mucosa.

38. The method of claim 37, wherein the wound is a post-operative lesion of the sinonasal mucosa following endoscopic sinus surgery.

39. The method of claim 37 or claim 38, wherein the composition or device is administered intraoperatively following endoscopic sinus surgery.

40. The method of any one of claims 24 to 39, comprising administering the composition or device intranasally to the subject.

41. The composition of any one of claims 1 to 16 or the device of any one of claims 17 to 21 for use in treating a condition associated with inflammation in a subject in need thereof.

42. The composition or the device for use of claim 41 for treating a condition associated with inflammation of the nasal mucosa.

43. The composition or the device for use of claim 41 or claim 42, wherein the composition or device is formulated for intranasal administration to the subject in need thereof.

44. The composition of any one of claims 1 to 16 or the device of any one of claims 17 to 21 for use in promoting wound healing in a subject in need thereof.

45. The composition or the device for use of claim 44, wherein the wound is a wound of the nasal mucosa.

46. The composition or the device for use of claim 44 or claim 45, wherein the composition is formulated, and the device is configured, for intranasal administration to the subject in need thereof.

47. Use of a therapeutically effective combination of (i) cromoglycic acid, or a pharmaceutically-acceptable salt thereof, and (ii) hyaluronic acid, or a pharmaceutically-acceptable salt thereof (HA) in the manufacture of a medicament for treating a condition associated with inflammation in a subject in need thereof.

48. The use of claim 47, wherein the condition associated with inflammation is a condition associated with inflammation of the nasal mucosa.

49. The use of claim 47 or claim 48, wherein the medicament is formulated for intranasal administration to the subject in need thereof.

50. The use of any one of claims 47 to 49, wherein the condition is associated with inflammation of the sinonasal mucosa.

51. The use of claim 50, wherein the condition is chronic rhinosinusitis (CRS).

52. The use of claim 51, wherein the CRS is chronic rhinosinusitis with nasal polyps (CRSwNP) or chronic rhinosinusitis without nasal polyps (CRSsNP).

53. The use of claim 52, wherein the CRS is chronic rhinosinusitis with nasal polyps (CRSwNP).

54. The use of any one of claims 47 to 53, wherein the composition is formulated for administration intraoperatively following endoscopic sinus surgery.

55. Use of a therapeutically effective combination of (i) cromoglycic acid, or a pharmaceutically-acceptable salt thereof, and (ii) hyaluronic acid, or a pharmaceutically-acceptable salt thereof (HA) in the manufacture of a medicament for promoting wound healing in a subject in need thereof.

56. The use of claim 55, wherein the wound is a wound of the nasal mucosa.

57. The use of claim 55 or claim 56, wherein the medicament is formulated for intranasal administration to the subject in need thereof.
